(19) United States
(12) Patent Application Publication
(10) Pub. No.: US 2026/0097049 A1
(43) Pub. Date: Apr. 9, 2026

(54) CANNABINOID ANALOGS AND METHODS OF USE FOR TREATMENT AND PREVENTION OF CANCER

(71) Applicant: BlackStone Therapeutics, LLC, Parker, CO (US)

(72) Inventors: Westley CRUCES, Parker, CO (US); Kyle P. RAY, Parker, CO (US); Prakash JAGTAP, Parker, CO (US)

(73) Assignee: BlackStone Therapeutics, LLC, Parker, CO (US)

(21) Appl. No.: 19/115,168

(22) PCT Filed: Sep. 29, 2023
(86) PCT No.: PCT/IB2023/059811

§ 371 Date: Mar. 25, 2025

Related U.S. Application Data
(60) Provisional application No. 63/411,506, filed on Sep. 29, 2022.
(60) Provisional application No. 63/487,547, filed on Feb. 28, 2023.

(51) Int. Cl. A61K 31/00; A61K 31/131; A61K 31/175; A61K 31/196; A61K 31/198; A61K 31/255; A61K 31/337; A61K 31/495; A61K 31/513; A61K 31/675; A61K 31/7068; A61P 35/00; C07C 39/17; C07D 311/80

(52) U.S. Cl. CPC A61K 31/658; A61K 31/131; A61K 31/175; A61K 31/196; A61K 31/198; A61K 31/255; A61K 31/337; A61K 31/495; A61K 31/513; A61K 31/675; A61K 31/7068; A61P 35/00; C07C 39/17; C07D 311/80

(57) ABSTRACT

Hexahydrocannabinol (HHC), hexahydrocannabidiol (H4CBD), and hexahydrocannabidivarin (H4CBDV) derivatives are disclosed. Also disclosed are pharmaceutical compositions comprising such derivatives. Further described are methods of use thereof for treatment and prevention of cancer, including pancreatic and lung cancer.

### CROSS REFERENCE TO RELATED APPLICATIONS

This application claims the benefit of priority of U.S. Provisional Patent Application No. 63/411,506 filed Sep. 29, 2022, and U.S. Provisional Patent Application No. 63/487,547 filed Feb. 28, 2023, all of which are hereby incorporated by reference in their entirety.

### BACKGROUND

### I. Field of the Invention

Aspects of this invention relate to the fields of organic chemistry and medicinal chemistry.

### II. Background

Hydrogenated Cannabinoids are semi-synthetic cannabinoids made from naturally occurring cannabinoids such as tetrahydrocannabinol (THC), cannabidiol (CBD) and cannabidivarin (CBDV), some which have been demonstrated to produce anti-cancer properties towards the inhibition of tumor angiogenesis and cell proliferation. Particular hydrogenated cannabidiols, such as hexahydrocannabinol (HHC), hexahydrocannabidiol (THD/H4CBD), hexahydrocannabidivarin (H4CBDV) and related parent scaffolds, have also been shown to have anti-cancer properties in vitro. There remains a need for synthesis and characterization, including biological characterization, of additional hydrogenated cannabinoids, such as those that cannot be synthesized directly from the naturally occurring cannabinoids, as well as therapeutic and prophylactic methods of use thereof such as in the treatment or prevention of cancer.

### SUMMARY

Aspects of the present disclosure are based, at least in part, on the synthesis and characterization of derivatives of hexahydrocannabinol (HHC), hexahydrocannabidiol (H4CBD) and hexahydrocannabidivarin (H4CBDV) with various functional moieties. As disclosed herein, these compounds were tested for cytotoxicity against various cancer cell lines, including pancreatic and lung carcinoma cells. Accordingly, disclosed herein, in some aspects, are novel hexahydrocannabinol derivatives. Further disclosed are methods of use thereof in treatment and prevention of cancer.

Some aspects of the disclosure are directed to a compound of formula (I) or formula (II):

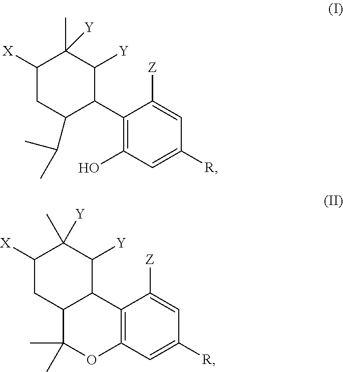

where
X is H, halogen, CF₃, or OR²; R² is CF₃, or C₁₋₆ alkyl; Y is H, or two adjacent Y are taken together to form a 3-membered heterocylcoalkyl ring, or two adjacent Y are taken together to form a 3-membered cycloalkyl ring optionally substituted with two R³; each R³ is independently H or F; Z is OR⁴; R⁴ is H, C₁₋₉ substituted or unsubstituted alkyl, C₁₋₉ substituted or unsubstituted alkenyl, C₁₋₉ substituted or unsubstituted alkynyl, OC(O)(CH₂)ₙC(O)—O-gemcitabine, or OC(O)(CH═CH)ₙC(O)—O-gemcitabine, wherein n is an integer from 1-20; R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁵; and R⁵ is H, C₁₋₉ substituted or unsubstituted alkyl, C₁₋₉ substituted or unsubstituted alkenyl, or C₁₋₉ substituted or unsubstituted alkynyl.

In some aspects, the compound is further defined as:

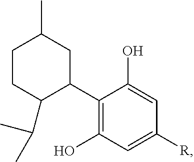

wherein R is a substituted or unsubstituted alkyl group ranging from 1 to 9 carbon atoms.

In some aspects, R is CH₃, CH₂CH₃, CH₂CH₂CH₃, CH(CH₃)₂, CH₂CH₂CH₂CH₃, C(CH₃)₃, CH₂CH₂CH₂CH₂CH₃, CH₂CH₂CH₂CH₂CH₂CH₃, CH₂CH₂CH₂CH₂CH₂CH₂CH₃, CH(CH₃)CH(CH₃)CH₂CH₂CH₂CH₂CH₃, C(CH₃)₂CH₂CH₂CH₂CH₂CH₂CH₃, CH₂CH₂CH₂CH₂CH₂CH₂CH₂CH₃, CH₂CH₂CH₂CH₂CH₂CH₂CH₂CH₂CH₃, CH(CH₃)₂, CH₂CH(CH₃)₂, or CH₂CH₂CH(CH₃)₂.

In some aspects, the compound is further defined as

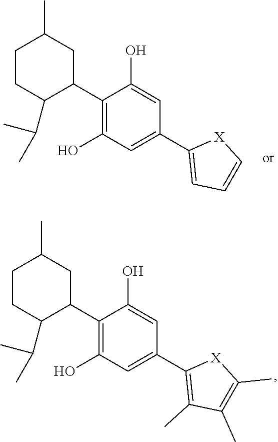

wherein X is N, NCH₃, O, or S.

In some aspects, the compound is further defined as

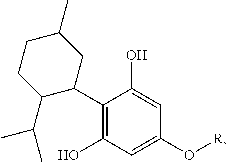

wherein R is C₁₋₄ alkyl-CH═CF₂.

In some aspects, the compound is further defined as

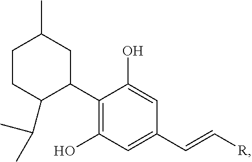

wherein R is a substituted or unsubstituted alkyl group ranging from 1 to 7 carbon atoms.

In some aspects, the compound is further defined as

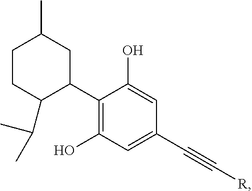

wherein R is a substituted or unsubstituted alkyl group ranging from 1 to 7 carbon atoms.

In some aspects, the compound is further defined as

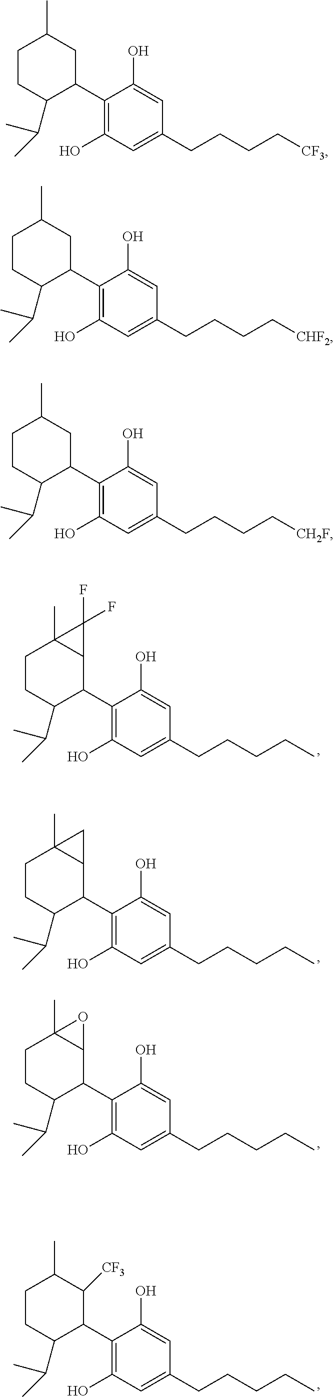

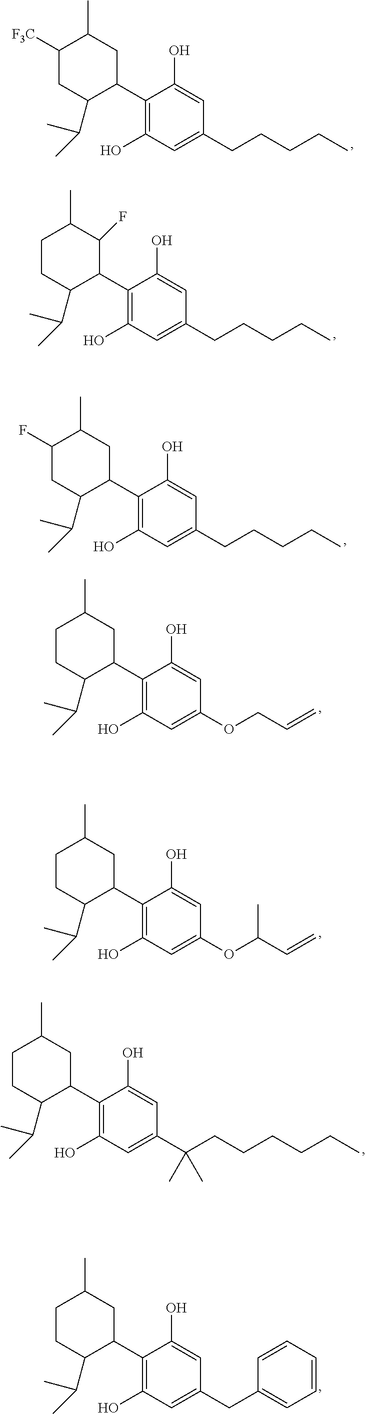

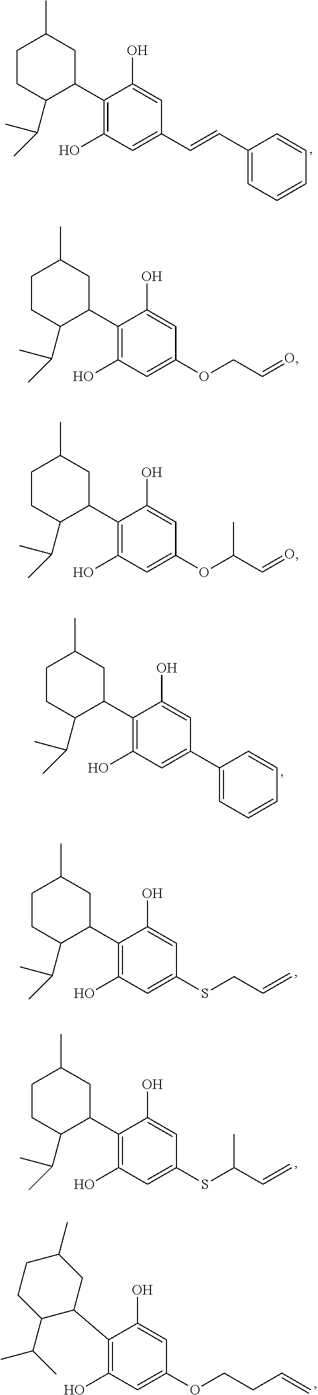

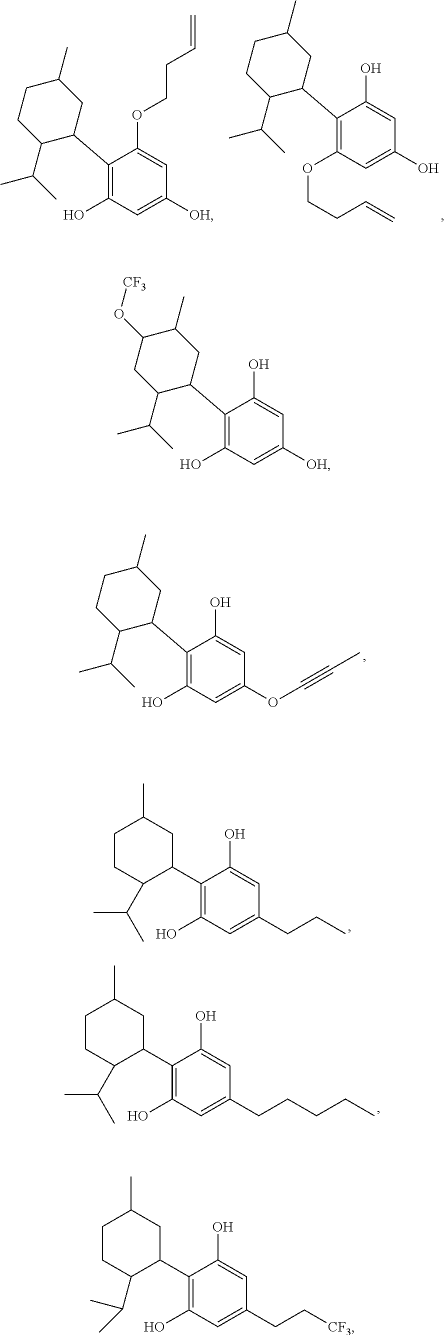

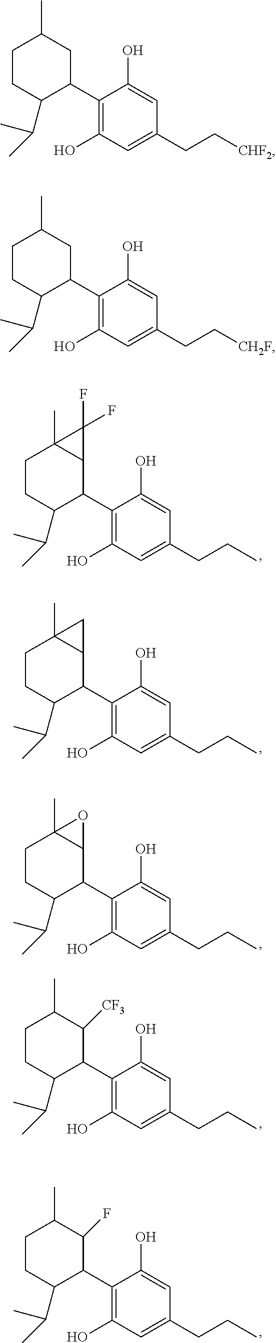

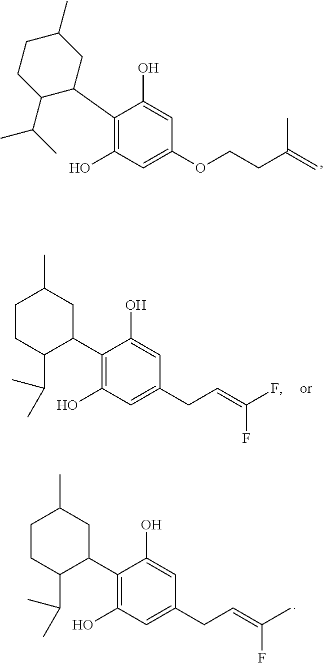

Any one or more of the preceding compounds may be specifically excluded in certain embodiments.

Some aspects of the disclosure are directed to a pharmaceutical composition comprising a compound disclosed herein or a derivative, or diastereomer, or enantiomer, or stereoisomer thereof. In some aspects, the pharmaceutical composition further comprises a pharmaceutically acceptable excipient. In some aspects, the pharmaceutical composition further comprises at least one additional chemotherapeutic. The at least one additional chemotherapeutic may comprise one or more of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride, Olaparib, veliparib, rucaparib, talazoparib, or niraparib. In some aspects, the chemotherapeutic is covalently linked to the compound of Formula I or Formula II. In some aspects, the chemotherapeutic is not covalently linked to the compound of Formula I or Formula II. In some aspects, the chemotherapeutic is covalently linked to the compound of Formula I or Formula II via a linker. In some aspects, the linker is an ester or amino acid linker. Various chemotherapeutics are recognized in the art and contemplated herein. In some aspects, the chemotherapeutic is gemcitabine, paclitaxel, or 5-fluorouracil. In some aspects, the pharmaceutical composition comprises a compound having formula:

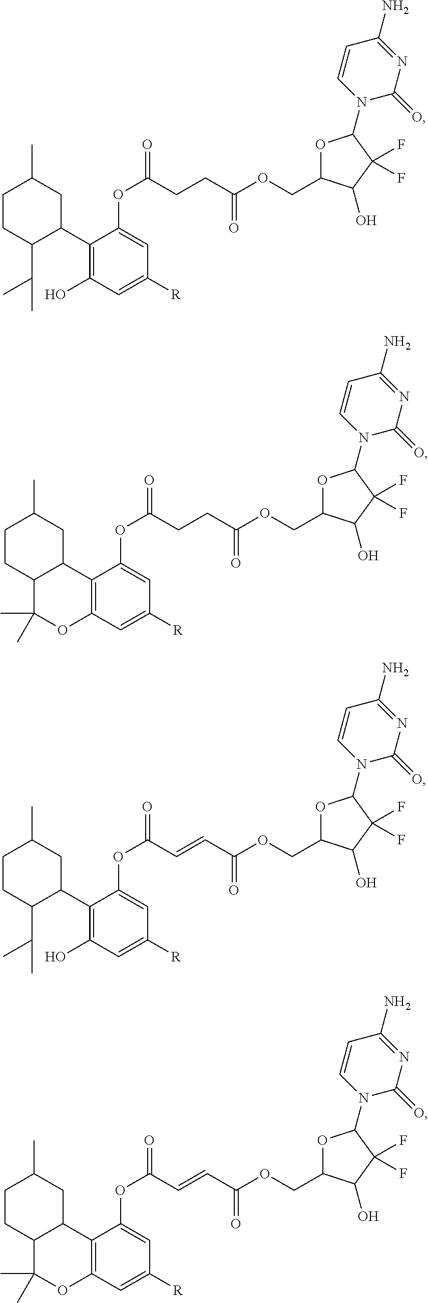

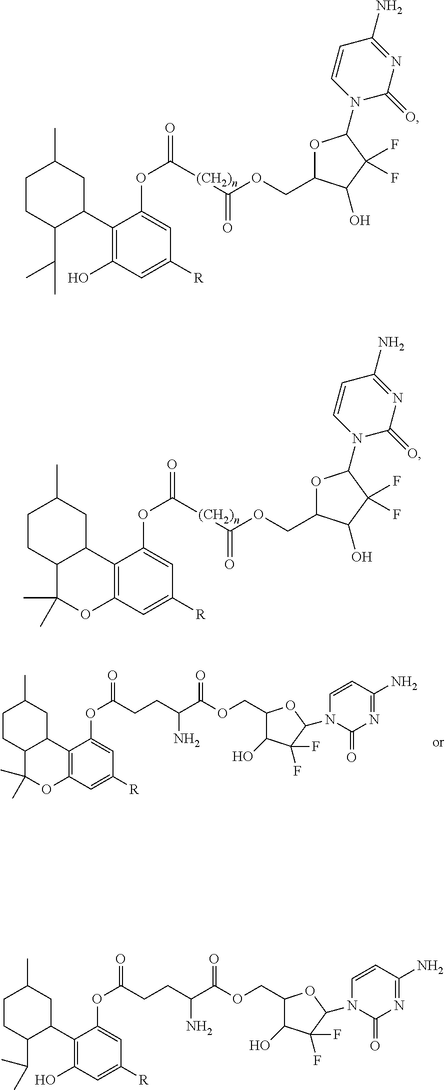

wherein n is an integer between 1 and 10; R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁶; and R⁶ is H, C₁₋₉ substituted or unsubstituted alkyl, C₁₋₉ substituted or unsubstituted alkenyl, or C₁₋₉ substituted or unsubstituted alkynyl.

Some aspects of the disclosure are directed to a method of treating cancer comprising administering to a subject a therapeutically effective amount of a pharmaceutical composition described herein. In some aspects, the subject has pancreatic cancer. In some aspects, the subject has lung cancer. In some aspects, a cancer cell is from a cancer selected from the group consisting of: multiple myeloma, leukemia, alveolar rhabdomyosarcoma, melanoma, lymphoma, astrocytoma, biphasic synovial sarcoma, bladder carcinoma, bone cancer, breast cancer, cecum adenocarcinoma, cervical cancer, cns cancer, colon cancer, colorectal cancer, duodenal adenocarcinoma, embryonal rhabdomyosarcoma, endometrial cancer, epithelioid sarcoma, fibrosarcoma, gastric cancer, signet ring cell gastric adenocarcinoma, gestational choriocarcinoma, glioblastoma, hereditary thyroid gland medullary carcinoma, hypopharyngeal squamous cell carcinoma, invasive ductal carcinoma, liposarcoma, lung cancer, neuroblastoma, osteosarcoma, ovarian cancer, uterine cancer, pancreatic cancer, papillary renal cell carcinoma, prostate cancer, rectal adenocarcinoma, medulloblastoma, renal cancer, testicular embryonal carcinoma, and tongue squamous cell carcinoma. Also disclosed are methods for killing cancer cells comprising administering to the cancer cells an effective amount of a compound or pharmaceutical composition disclosed herein.

Some aspects of the disclosure are directed to a pharmaceutical composition comprising a compound as disclosed herein. The terms delta-8 tetrahydrocannabinol, delta-8 THC, D8 THC, Δ8-tetrahydrocannabinol, and Δ8-THC are used interchangeably herein. The terms delta-9 tetrahydrocannabinol, delta-9 THC, D9 THC, Δ9-tetrahydrocannabinol, Δ9-THC, and THC are used interchangeably herein. The terms tetrahydrocannabivarin, THCV, D9-THCV, and D8-THCV are used interchangeably herein. H₄CBD, and THD are used interchangeably herein. The terms hexahydrocannabidivarin, and H4CBDV are used interchangeably herein. The terms hexahydrocannabinol and HHC are used interchangeably herein. The term “semi-synthetic” is defined as a method that employs natural compounds or compounds derived from natural compounds as starting materials to produce different compounds.

Some aspects relate to methods of synthesizing any of the compounds disclosed herein, such as any of the hydrogenated cannabinoids disclosed herein. In some aspects, the method comprises saturating, such as through hydrogenation, a double bond in a cannabinoid scaffold. The cannabinoid scaffold may be derived from hemp, Cannabis sativa, Cannabis indica, Echinacea purpurea, Echinacea angustifolia, Acmella oleracea, Helichrysum umbraculigerum, and/or Radula marginata, or any other source containing a cannabinoid scaffold. In some aspects, the cannabinoid scaffold comprises cannabinol, tetrahydrocannabinol, cannabidiol, and/or cannabidivarin.

In the context of the present invention, at least the following 42 aspects are described. Aspect 1 includes a compound of formula I or formula II,

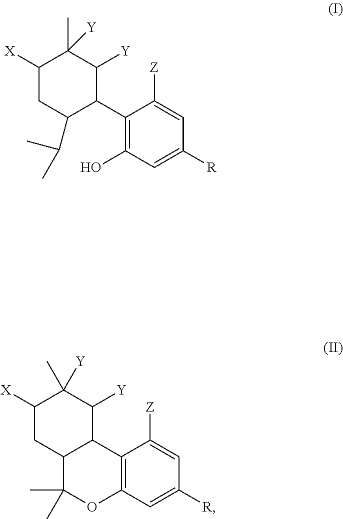

wherein X is H, halogen, CF₃, or OR²; R² is CF₃, or C₁₋₆ alkyl; Y is H, or two adjacent Y are taken together to form a 3-membered heterocylcoalkyl ring, or two adjacent Y are taken together to form a 3-membered cycloalkyl ring optionally substituted with two R³; each R³ is independently H or F; Z is OR⁴; R⁴ is H, C₁₋₉ substituted or unsubstituted alkyl, C₁₋₉ substituted or unsubstituted alkenyl, C₁₋₉ substituted or unsubstituted alkynyl, OC(O)(CH₂)ₙC(O)—O-gemcitabine, or OC(O)(CH═CH)ₙC(O)—O-gemcitabine, wherein n is an integer from 1-20; R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁵; and R⁵ is H, C₁₋₉ substituted or unsubstituted alkyl, C₁₋₉ substituted or unsubstituted alkenyl, or C₁₋₉ substituted or unsubstituted alkynyl. Aspect 2 depends on Aspect 1, wherein the compound is further defined as

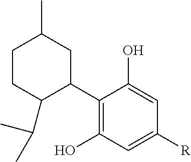

wherein R is a substituted or unsubstituted alkyl group ranging from 1 to 9 carbon atoms. Aspect 3 depends on Aspect 1 or Aspect 2, wherein R is CH₃. Aspect 4 depends on Aspect 1 or Aspect 2, wherein R is CH₂CH₃. Aspect 5 depends on Aspect 1 or Aspect 2, wherein R is CH₂CH₂CH₃. Aspect 6 depends on Aspect 1 or Aspect 2, wherein R is CH₂CH₂CH₂CH₃. Aspect 7 depends on Aspect 1 or Aspect 2, wherein R is CH₂CH₂CH₂CH₂CH₃. Aspect 8 depends on Aspect 1 or Aspect 2, wherein R is CH₂CH₂CH₂CH₂CH₂CH₃. Aspect 9 depends on Aspect 1 or Aspect 2, wherein R is CH₂CH₂CH₂CH₂CH₂CH₂CH₃. Aspect 10 depends on Aspect 1 or Aspect 2, wherein R is CH₂CH₂CH₂CH₂CH₂CH₂CH₂CH₃. Aspect 11 depends on Aspect 1 or Aspect 2, wherein R is CH₂CH₂CH₂CH₂CH₂CH₂CH₂CH₂CH₃. Aspect 12 depends on Aspect 1 or Aspect 2, wherein R is C(CH₃)₃. Aspect 13 depends on Aspect 1 or Aspect 2, wherein R is CH(CH₃)CH(CH₃)CH₂CH₂CH₂CH₂CH₃. Aspect 14 depends on Aspect 1 or Aspect 2, wherein R is CH(CH₃)₂. Aspect 15 depends on Aspect 1 or Aspect 2, wherein R is CH₂CH(CH₃)₂. Aspect 16 depends on Aspect 1 or Aspect 2, wherein R is CH₂CH₂CH(CH₃)₂. Aspect 17 depends on Aspect 1 or Aspect 2, wherein R is C(CH₃)₂CH₂CH₂CH₂CH₂CH₂CH₃. Aspect 18 depends on Aspect 1, wherein the compound is further defined as

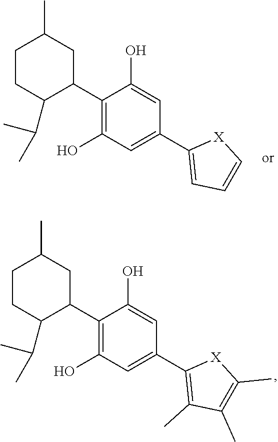

wherein X is N, O, or S. Aspect 19 depends on Aspect 1, wherein the compound is further defined as

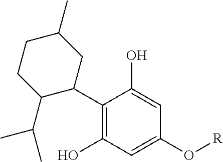

wherein R is C₁₋₄ alkyl-CH═CF₂. Aspect 20 depends on Aspect 1, wherein the compound is further defined as

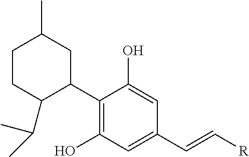

wherein R is a substituted or unsubstituted alkyl group ranging from 1 to 7 carbon atoms. Aspect 21 depends on Aspect 1, wherein the compound is further defined as

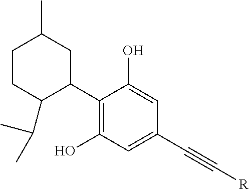

wherein R is a substituted or unsubstituted alkyl group ranging from 1 to 7 carbon atoms. Aspect 22 depends on Aspect 1, wherein the compound is further defined as

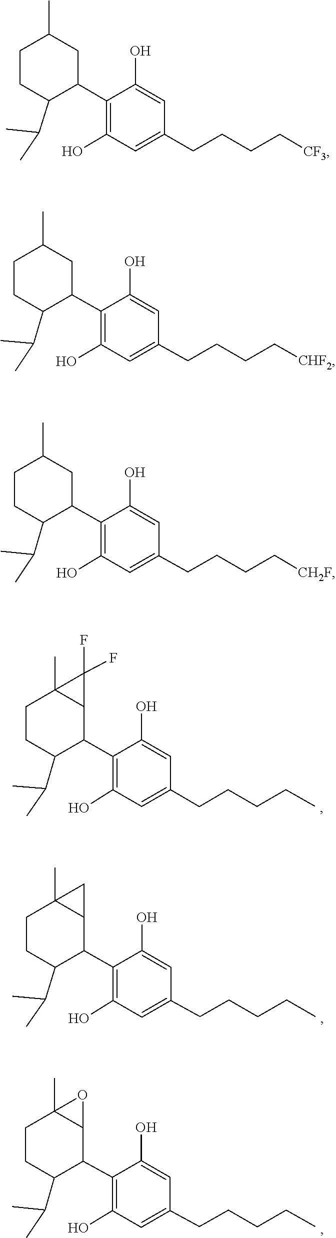

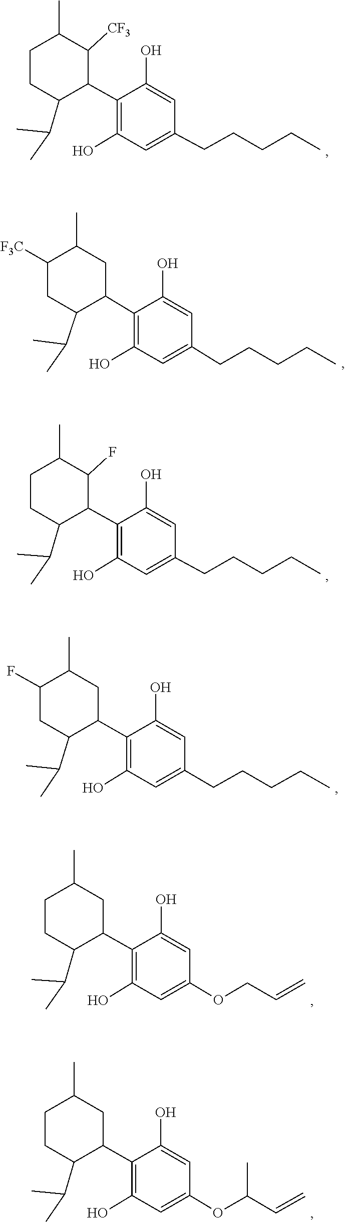

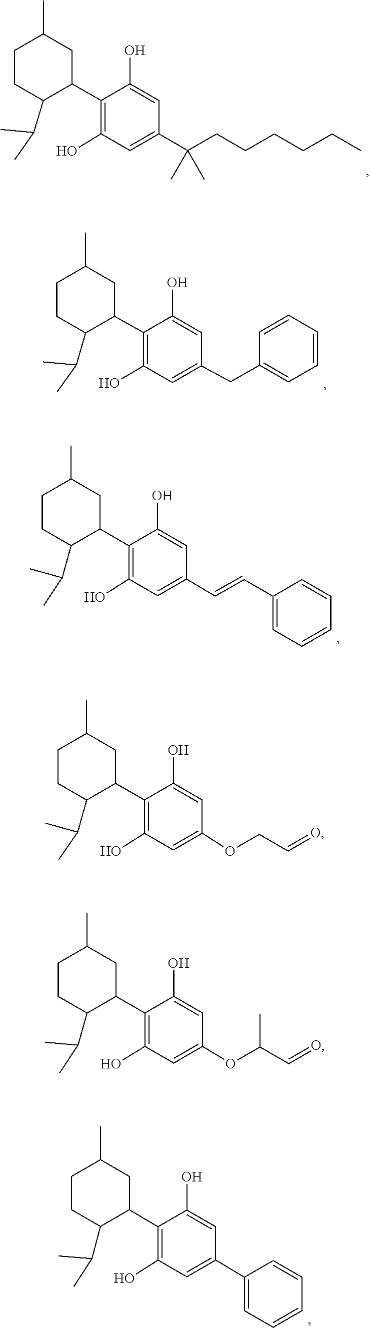

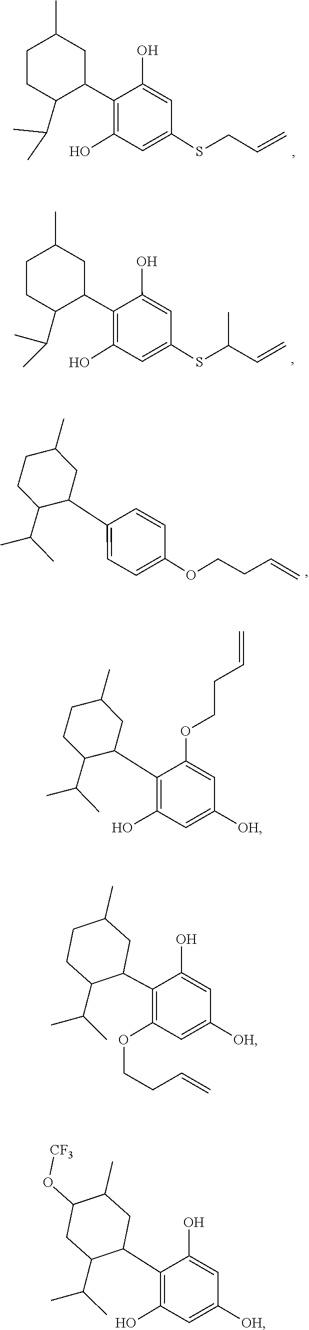

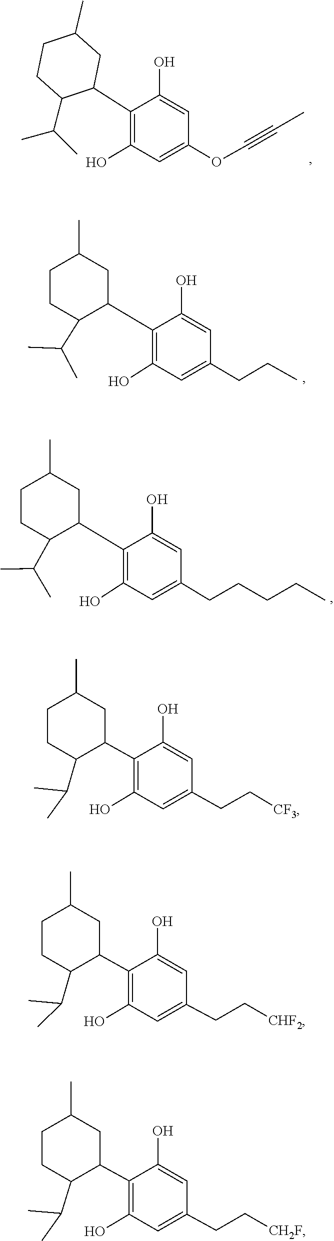

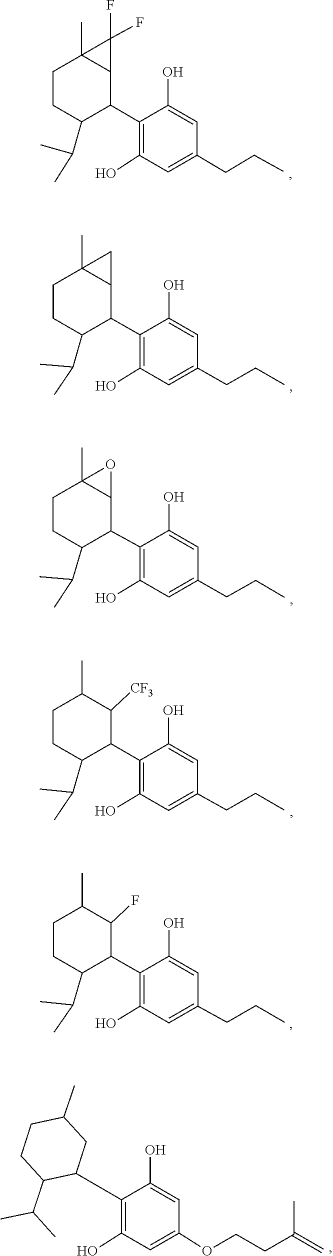

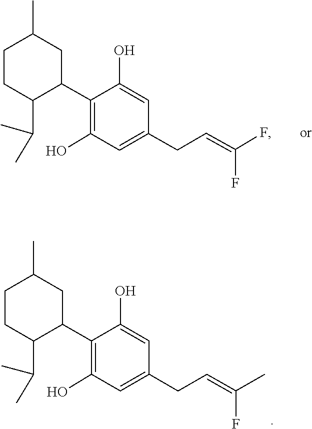 [SMILES: CC1CCC(C(C)C)C(c2c(O)cc(CC=C(F)F)cc2O)C1 ; C/C(F)=C/Cc1cc(O)c(C2CC(C)CCC2C(C)C)c(O)c1]

Aspect 23 includes a pharmaceutical composition comprising the compound of any of Aspects 1 to Aspect 22, or a derivative or a diastereomer or enantiomer or stereoisomer thereof. Aspect 24 depends on Aspect 23 further comprising a pharmaceutically acceptable excipient. Aspect 25 depends on Aspect 23 or Aspect 24, further comprising at least one additional chemotherapeutic. Aspect 26 depends on Aspect 25, wherein the at least one additional chemotherapeutic comprises one or more of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride, Olaparib, veliparib, rucaparib, talazoparib, or niraparib. Aspect 27 depends on Aspect 25 or Aspect 26, wherein the at least one additional chemotherapeutic is covalently linked to the compound of Formula I or Formula II. Aspect 28 depends on Aspect 27, wherein the at least one additional chemotherapeutic is covalently linked to the compound of Formula I or Formula II via a linker. Aspect 29 depends on Aspect 28, wherein the linker is an ester or amino acid linker. Aspect 30 depends on any one of Aspect 23 to Aspect 29, wherein the at least one additional chemotherapeutic is gemcitabine, paclitaxel, or 5-fluorouracil. Aspect 31 depends on any one of Aspect 23 to Aspect 30, wherein the pharmaceutical composition comprises a compound having formula

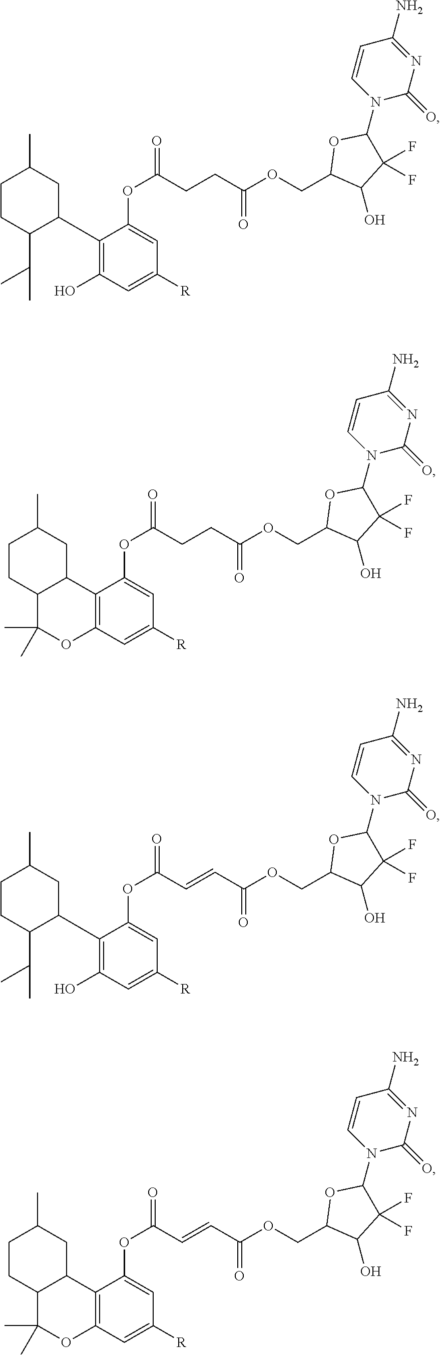

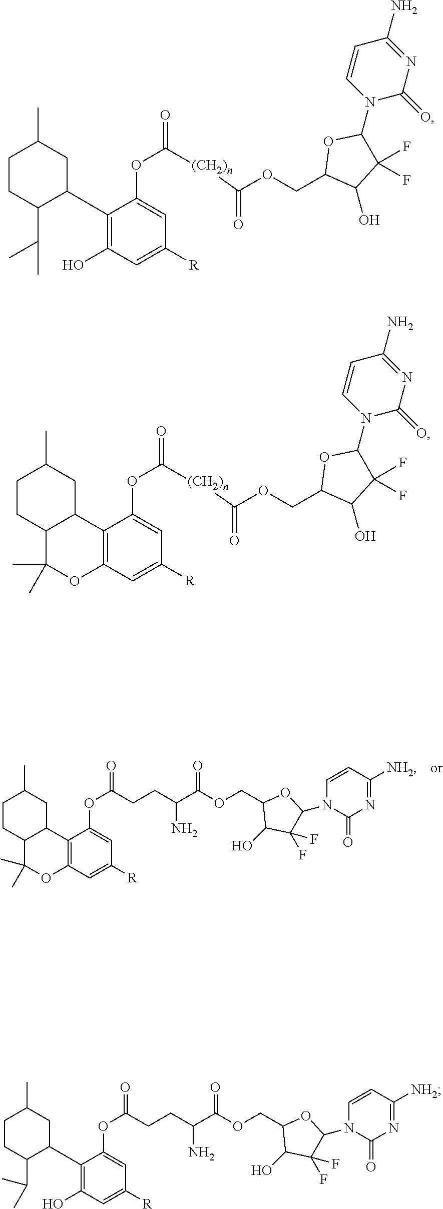

wherein n is an integer between 1 and 10; wherein R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁶; and wherein R⁶ is H, C₁₋₉ substituted or unsubstituted alkyl, C₁₋₉ substituted or unsubstituted alkenyl, or C₁₋₉ substituted or unsubstituted alkynyl. Aspect 32 is a method of treating cancer comprising administering to a subject a therapeutically effective amount of a pharmaceutical composition comprising the compound of any of Aspect 1 to Aspect 22 or the pharmaceutical composition of any of Aspect 23 to Aspect 31. Aspect 33 depends on Aspect 32, wherein the subject has pancreatic cancer. Aspect 34 depends on Aspect 32, wherein the subject has lung cancer. Aspect 35 is a method of killing a cancer cell, the method comprising administering to the cancer cell an effective amount of the compound of any of Aspect 1 to Aspect 22 or an effective amount of the pharmaceutical composition of any of Aspect 23 to Aspect 31. Aspect 36 depends on Aspect 35, wherein the cancer cell is a pancreatic cancer cell. Aspect 37 depends on Aspect 35, wherein the cancer cell is a lung cancer cell. Aspect 38 depends on Aspect 35, wherein the cancer cell is from a cancer selected from the group consisting of: multiple myeloma, leukemia, alveolar rhabdomyosarcoma, melanoma, lymphoma, astrocytoma, biphasic synovial sarcoma, bladder carcinoma, bone cancer, breast cancer, cecum adenocarcinoma, cervical cancer, cns cancer, colon cancer, colorectal cancer, duodenal adenocarcinoma, embryonal rhabdomyosarcoma, endometrial cancer, epithelioid sarcoma, fibrosarcoma, gastric cancer, signet ring cell gastric adenocarcinoma, gestational choriocarcinoma, glioblastoma, hereditary thyroid gland medullary carcinoma, hypopharyngeal squamous cell carcinoma, invasive ductal carcinoma, liposarcoma, lung cancer, neuroblastoma, osteosarcoma, ovarian cancer, uterine cancer, pancreatic cancer, papillary renal cell carcinoma, prostate cancer, rectal adenocarcinoma, medulloblastoma, renal cancer, testicular embryonal carcinoma, and tongue squamous cell carcinoma. Aspect 39 is a method of synthesizing the compound of any one of claims 1-22, the method comprising saturating a double bond between carbons 9 and 10 in a cannabinoid scaffold. Aspect 40 depends on Aspect 39, wherein the cannabinoid scaffold is derived from hemp, Cannabis sativa, Cannabis indica, Echinacea purpurea, Echinacea angustifolia, Acmella oleracea, Helichrysum umbraculigerum, and/or Radula marginata. Aspect 41 depends on Aspect 39 or Aspect 40, wherein the cannabinoid scaffold comprises cannabinol, tetrahydrocannabinol, cannabidiol, and/or cannabidivarin. Aspect 42 depends on Aspect 39, wherein the cannabinoid scaffold comprises cannabinol, tetrahydrocannabinol, cannabidiol, and/or cannabidivarin, wherein the scaffold is isolated from hemp, Cannabis sativa, Cannabis indica, Echinacea purpurea, Echinacea angustifolia, Acmella oleracea, Helichrysum umbraculigerum, and/or Radula marginata.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is the chemical structure of compound H4CBD, (9R)-H4CBD, (9S)-H4CBD, H4CBDV, HHC, (9R)-HHC, and (9S)-HHC.

FIG. 2 demonstrates the use of H4CBD to evaluate viability in cancer cells. 9R=9R-Hexahydrocannabinol. 9S=9S-Hexahydrocannabinol. THCV=D9/D8-THCV.

FIG. 3 demonstrates the use of H4CBD to evaluate viability in cancer cells. 9R/9S is 9R/9S-Hexahydrocannabinol (HHC). THCV=D9/D8-THCV

FIG. 4 demonstrates the use of H4CBD to evaluate viability in cancer cells.

FIG. 5 demonstrates the use of H4CBD to evaluate viability in cancer cells.

FIG. 6 is an HPLC trace of the enantiomers of H4CBD obtained by methods disclosed herein.

FIG. 7 is a mass spectrum of (R)-H4CBD and includes prominent ions at m/z 193, 233, and 318.

FIG. 8 is a mass spectrum of (S)-H4CBD and includes prominent ions at m/z 193, 233, and 318.

FIG. 9 is a mass spectrum of H4CBD and includes prominent ions at m/z 193, 233, and 318.

FIG. 10 demonstrates the percent viability of PANC-1 cells treated with the indicated compound at the indicated concentration. RH4=(R)-H4CBD, SH4=(S)-H4CBD, H4CBBV=H4CBDV.

FIG. 11 demonstrates the percent viability of Mia PaCa2 cells treated with the indicated compound at the indicated concentration. RH4=(R)-H4CBD, SH4=(S)-H4CBD, H4CBBV=H4CBDV.

FIG. 12 demonstrates the percent viability of AsPC-1 cells treated with the indicated compound at the indicated concentration. RH4=(R)-H4CBD, SH4=(S)-H4CBD, H4CBBV=H4CBDV.

FIG. 13 demonstrates the comparison of effects of cannabinoid compounds on PANC1 and HPAF-II cells. 9R HHC=(R)-HHC, 9S HHC=(S)-HHC.

FIG. 14 demonstrates the comparison of effects of PARP inhibitors on Mia PaCa2 and HPAF-II cells.

FIGS. 15A-15B Exemplary reaction scheme. FIG. 15A demonstrates synthesis of gem-difluorocyclopropanes in a phase-transfer catalyzed system. FIG. 15B is a target compound example of the reaction scheme.

FIGS. 16A-16B Exemplary reaction scheme. FIG. 16A demonstrates insertion reactions of 1,2,2-trifluoroethylidene into carbon-hydrogen bonds of alkanes. FIG. 16B is a target compound example of the reaction scheme.

FIGS. 17A-17B Exemplary reaction scheme. FIG. 17A demonstrates the Simmons-Smith cyclopropanation reaction. FIG. 17B is a target compound example of the reaction scheme.

FIGS. 18A-18B Exemplary reaction scheme. FIG. 18A demonstrates oxidation of alkenes with H₂O₂ by an in-situ prepared Mn(II)/Pyridine-2-carboxylic acid catalyst. FIG. 18B is a target compound example of the reaction scheme.

FIGS. 19A-19B Exemplary reaction scheme. FIG. 19A demonstrates switchable 2,2,2-trifluoroethylation and gem-difluorovinylation of organoboronic acids with 2,2,2-trifluorodiazoethane. FIG. 19B is a target compound example of the reaction scheme.

FIGS. 20A-20B Exemplary reaction scheme. FIG. 20A demonstrates catalytic hydrotrifluoromethylation of styrenes and unactivated aliphatic alkenes via an organic photoredox system. FIG. 20B is a target compound example of the reaction scheme.

FIGS. 21A-21B Exemplary reaction scheme. FIG. 21A demonstrates third generation radical fluorinating agents based on N-fluoro-N-arylsulfonamides. FIG. 21B is a target compound example of the reaction scheme.

FIGS. 22A-22B Exemplary reaction scheme. FIG. 22A demonstrates quinoline-directed acyl C—O bond activation and alkene oxyacylation reactions. FIG. 22B is a target compound example of the reaction scheme.

FIGS. 23A-23B Exemplary reaction scheme. FIG. 23A demonstrates palladium-catalyzed etherification of allyl alcohols using phenols in the presence of titanium(IV) isopropoxide. FIG. 23B is a target compound example of the reaction scheme.

FIGS. 24A-24B Exemplary reaction scheme. FIG. 24A demonstrates synthesis of phenyl substituted side-chain analogues of classical cannabinoids. FIG. 24B is a target compound example of the reaction scheme.

FIGS. 25A-25B Exemplary reaction scheme. FIG. 25A demonstrates synthesis of preparing analogs via Julia Kocienski olefination. FIG. 25B is a target compound example of the reaction scheme.

FIGS. 26A-26B Exemplary reaction scheme. FIG. 26A demonstrates synthesis of preparing analogs via composite metal oxide solid alkali catalysis. FIG. 26B is a target compound example of the reaction scheme.

FIGS. 27A-27B Exemplary reaction scheme. FIG. 27A demonstrates enantioselective hydrogenations of esters with dynamic kinetic resolution. FIG. 27B is a target compound example of the reaction scheme.

FIGS. 28A-28B Exemplary reaction scheme. FIG. 28A demonstrates trapping of protonated phenols (ethers) by aromatics in super acids. FIG. 28B is a target compound example of the reaction scheme.

FIGS. 29A-29B Exemplary reaction scheme. FIG. 29A demonstrates a route to 5-substituted resorcinols and related systems. FIG. 29B is a target compound example of the reaction scheme.

FIGS. 30A-30B Exemplary reaction scheme. FIG. 30A demonstrates highly gamma-regioselective substitutions in allyl halides by alkylzincs and applications to zinc-ene cyclizations and the synthesis of (R)-(+)-dihydro-alpha-ionone. FIG. 30B is a target compound example of the reaction scheme.

FIGS. 31A-31B Exemplary reaction scheme. FIG. 31A demonstrates 1,2-vinyl rearrangements of secondary and tertiary homoallyllithiums. FIG. 31B is a target compound example of the reaction scheme.

FIG. 32 demonstrates quinoline-directed acyl C—O bond activation and alkene oxyacylation reactions.

FIGS. 33A-33B Exemplary reaction scheme. FIG. 33A demonstrates synthesis of (E)-6-(1-alkenyl)-substituted β-resorcylic acid esters. FIG. 33B is a target compound example of the reaction scheme.

FIGS. 34A-34B Exemplary reaction scheme. FIG. 34A demonstrates generation of alkyne hexahydrocannabinoid derivatives. FIG. 34B is a target compound example of the reaction scheme.

FIGS. 35A-35B Exemplary reaction scheme. FIG. 35A demonstrates intermolecular bromotrifluoromethoxylation of alkenes. FIG. 35B is a target compound example of the reaction scheme.

FIGS. 36A-36B Exemplary reaction scheme. FIG. 36A demonstrates generation of ynol ethers. FIG. 36B is a target compound example of the reaction scheme.

FIGS. 37A-37B Exemplary reaction scheme. FIG. 37A demonstrates preparing trifluorovinyl aryl ether compound from dihalogenated trifluoroethane. FIG. 37B is a target compound example of the reaction scheme.

FIGS. 38A-38B Exemplary reaction scheme. FIG. 38A demonstrates co-catalyzed hydroarylation of unactivated olefins. FIG. 38B is a target compound example of the reaction scheme.

FIG. 39 demonstrates percent viability of Δ549 (ATCC) lung carcinoma cells treated with the indicated concentration of the indicated compounds. GR-X1=H4CBDV, GR-X2=(R)-H4CBD, GR-X3=(S)-H4CBD, GR-X4=HHC. HHC was the only compound that presented higher cell viability from 25 μM, the other compounds presented cytotoxicity (cell viability <50%) until concentration 6.25 μM.

FIG. 40 is a comparison of the effects of the indicated compounds on cell viability in lung carcinoma cells.

FIG. 41 is a comparison of cell viability from different control drugs.

FIG. 42 is a comparison of certain compounds disclosed herein with paclitaxel. All concentrations of such produced better cell viability than the control drug paclitaxel.

FIG. 43 is a certain compounds disclosed herein with 5-fluorouracil. 5-fluorouracil presented the best cell viability when compared to the other two drugs, gemcitabine and paclitaxel, in the study. 5-fluorouracil presented better cell viability until concentration 25 μM, and when the concentrations of compounds were reducing the cell viability presented same results. Comparing FIG. 42 to FIG. 43, paclitaxel has a higher cytotoxicity at a lower concentration than 5-flurouracil.

FIGS. 44-51 are exemplary reaction schemes for the synthesis of C3-HCBD (H4CBDV) analogs.

FIGS. 52A-52D and 53A-53B show the plasma concentration over time of CCL-104 and CCL-106, administered either IV or PO to mice.

FIG. 54 shows the growth of tumors over time from MiaPaca cells xenograft into SCID mice that are either untreated, treated with CCL-104 (PO or IV), or treated with CCL-106 (PO or IV).

FIG. 55 shows final tumors after treatment in the xenograft study.

FIG. 56 depicts the chemical structure of the cannabinoid Δ9-THC, which has a double bond between the carbon atoms labeled 9 and 10.

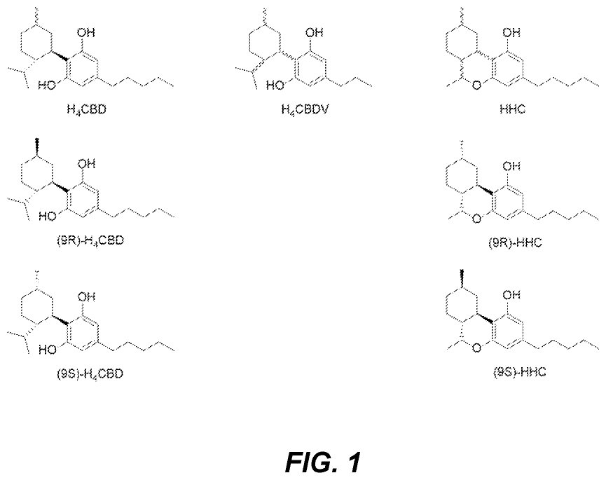

### DETAILED DESCRIPTION

### Chemical Definitions

The terms delta-8 tetrahydrocannabinol, delta-8 THC, D8 THC, Δ8-tetrahydrocannabinol, and Δ8-THC are used interchangeably herein. The terms delta-9 tetrahydrocannabinol, delta-9 THC, D9 THC, Δ9-tetrahydrocannabinol, Δ9-THC, and THC are used interchangeably herein. The terms tetrahydrocannabivarin, THCV, D9-THCV, and D8-THCV are used interchangeably herein. The terms hexahydrocannabinol and HHC are used interchangeably herein. The terms hexahydrocannabidiol, H₄CBD, and THD are used interchangeably herein. The terms hexahydrocannabidivarin, and H₄CBDV are used interchangeably herein. The term “semi-synthetic” is defined as a method that employs natural compounds or compounds derived from natural compounds as starting materials to produce different compounds.

The term “alkyl” includes straight-chain alkyl, branched-chain alkyl, cycloalkyl(alicyclic), cyclic alkyl, aryl-unsubstituted alkyl, aryl-substituted alkyl, heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. In some aspects, the term “alkyl group” denotes a monovalent linear or branched saturated hydrocarbon group of 1 to 12 carbon atoms. In certain embodiments, an alkyl group has 1 to 7 carbon atoms, and in more particular embodiments 1 to 4 carbon atoms. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —C(CH₃)₂(CH₂)₅CH₃ (dimethylheptyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —(CH₂)₂F, —(CH₂)₃F, —(CH₂)₄F, —(CH₂)₅F, —(CH₂)₆F, —(CH₂)₇F, —(CH₂)₈F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CH₂OC(O)CH₃, —CH₂CH₂NHCO₂C(CH₃)₃, and —CH₂Si(CH₃)₃. The term “aryl” refers to a radical, having a single carbon atom as a point of attachment, wherein the carbon atom is part of an aromatic ring structure containing only carbon atoms, 5 or more hydrogen atoms, and no heteroatoms. The phenyl and naphthalenyl groups are non-limiting examples of aryl groups. The benzyl group is a non-limiting example of an aryl-substituted alkyl group, where the alkyl group is methylene —CH₂— and the aryl group is a phenyl group.

The term “olefin” refers to a carbon-carbon double bond. The term “cyclohexyl” denotes a cyclized alkyl group having 6 carbon atoms. The term “cyclohexenyl” denotes a cyclized alkyl group having 6 carbon atoms and further having at least one nonaromatic carbon-carbon double bond. The phrase “cyclohexenyl olefin” refers to a carbon-carbon double bond or olefin of a cyclohexenyl ring.

The term “cyclohexyl” denotes a cyclized alkyl group having 6 carbon atoms. The term “cyclohexenyl” denotes a cyclized alkyl group having 6 carbon atoms and further having at least one nonaromatic carbon-carbon double bond.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Various forms of palladium catalyst useful for the reaction are discussed by Blaser et. al., Supported palladium catalysts for fine chemicals synthesis in Journal of Molecular Catalysis A: Chemical, 2001, v. 172, p. 3-18, the entirety of which is incorporated by reference.

In aspects disclosed herein, the compounds may be disclosed as a CCL identifier. The CCL identifier corresponds to the structures and cannabinoid name as shown in Table 1.

| CCL | | | |
|---|---|---|---|
| Number | Structure | Cannabinoid | MW |
| CCL-100 | 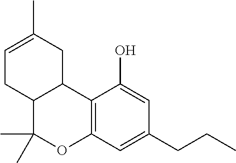 [SMILES: CCCc1cc(O)c2c(c1)OC(C)(C)C1CC=C(C)CC21] | THCV | 286.42 |
| CCL-101 | 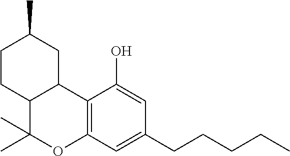 [SMILES: CCCCCc1cc(O)c2c(c1)OC(C)(C)C1CC[C@@H](C)CC21] | 9-(R)-HHC | 316.48 |
| CCL-102 | 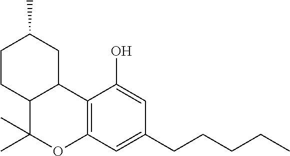 [SMILES: CCCCCc1cc(O)c2c(c1)OC(C)(C)C1CC[C@H](C)CC21] | 9-(S)-HHC | 316.48 |
| CCL-103 | 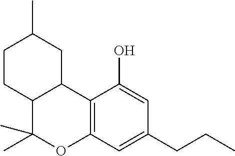 [SMILES: CCCc1cc(O)c2c(c1)OC(C)(C)C1CCC(C)CC21] | HHCV (racemate) | 288.42 |
| CCL-104 | 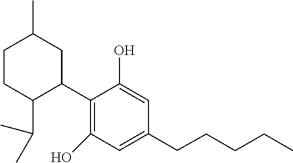 [SMILES: CCCCCc1cc(O)c(C2CC(C)CCC2C(C)C)c(O)c1] | H4CBD (racemate) | 318.49 |
| CCL-105 | 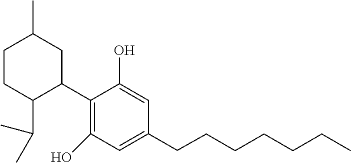 [SMILES: CCCCCCCc1cc(O)c(C2CC(C)CCC2C(C)C)c(O)c1] | H4CBDP (racemate) | 346.55 |
| CCL-106 | 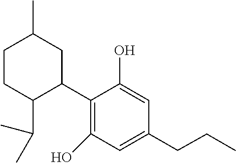 [SMILES: CCCc1cc(O)c(C2CC(C)CCC2C(C)C)c(O)c1] | H4CBDV (racemate) | 290.44 |
| CCL-107 | 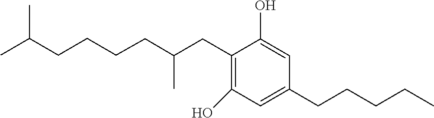 [SMILES: CCCCCc1cc(O)c(CC(C)CCCCC(C)C)c(O)c1] | HCBG (racemate) | 320.51 |
| CCL-108 | 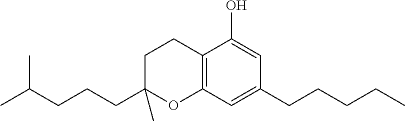 [SMILES: CCCCCc1cc(O)c2c(c1)OC(C)(CCCC(C)C)CC2] | HCBC (racemate) | 318.49 |
| CCL-109 | 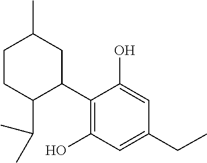 [SMILES: CCc1cc(O)c(C2CC(C)CCC2C(C)C)c(O)c1] | H4-C2CBD (racemate) | 276.42 |
| CCL-110 | 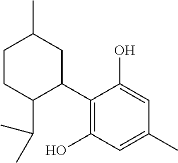 [SMILES: Cc1cc(O)c(C2CC(C)CCC2C(C)C)c(O)c1] | H4-C1-CBD (racemate) | 262.39 |
| CCL-111 | 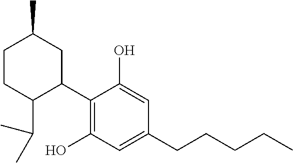 [SMILES: CCCCCc1cc(O)c(C2C[C@H](C)CCC2C(C)C)c(O)c1] | (R)-H4CBD | 318.5 |
| CCL-112 | 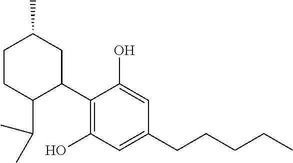 [SMILES: CCCCCc1cc(O)c(C2C[C@@H](C)CCC2C(C)C)c(O)c1] | (S)-H4CBD | 318.5 |
| CCL-113 | 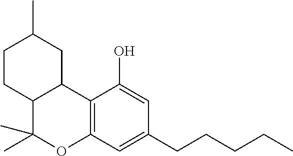 [SMILES: CCCCCc1cc(O)c2c(c1)OC(C)(C)C1CCC(C)CC21] | HHC (racemate) | 316.48 |
| CCL-114 | 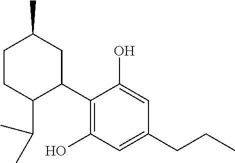 [SMILES: CCCc1cc(O)c(C2C[C@H](C)CCC2C(C)C)c(O)c1] | (R)-H4CBDV | 290.45 |
| CCL-115 | 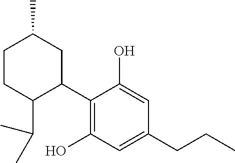 [SMILES: CCCc1cc(O)c(C2C[C@@H](C)CCC2C(C)C)c(O)c1] | (S)-H4CBDV | 290.45 |
| CCL-116 | 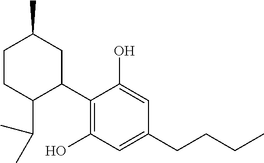 [SMILES: CCCCc1cc(O)c(C2C[C@H](C)CCC2C(C)C)c(O)c1] | (R)- H4C4CBD | 304.47 |
| CCL-117 | 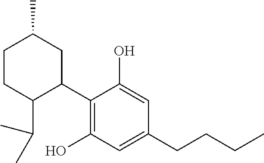 [SMILES: CCCCc1cc(O)c(C2C[C@@H](C)CCC2C(C)C)c(O)c1] | (S)-H4C4CBD | 304.47 |

### Therapeutic Methods

Certain aspects of the disclosure concern synthetic compounds, including those derived from cannabinoids, for selectively disrupting the replication of cancer cells. The synthetic compounds may be comprised in pharmaceutical compositions, which may be used in methods for the treatment of diseases, including cancer. Aspects of the disclosure are directed to compositions and methods for therapeutic use. The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of one or more symptoms or manifestation of a disease or condition. As an example, the disease or condition may be cancer, and the one or more symptoms may be, for example, symptoms associated with the cancer. For purposes of this disclosure, beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” does not necessarily indicate complete eradication or cure of the disease or condition, or associated symptoms thereof. Those in need of treatment include those already with the condition or disorder as well as those prone to have the condition or disorder or those in which the condition or disorder is to be prevented. The results of treatment can be determined by methods known in the art, such as determination of reduction of, e.g., tumor burden, determination of restoration of function, or other methods known in the art.

As used herein, “prevent,” and similar words such as “prevented,” “preventing,” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition. Prevention may be considered complete when onset of a disease, disorder, or condition has been delayed for a predefined period of time.

### A. Cancer Therapy

In some aspects, the disclosed methods comprise administering a cancer therapy to a subject or patient. The cancer therapy may be chosen based on expression level measurements, alone or in combination with a clinical risk score calculated for the subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a systemic cancer therapy. In some aspects, the cancer therapy comprises an immunotherapy, which may be an immune blockade or immune checkpoint inhibitor therapy. In some aspects, the cancer therapy comprises radiotherapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In particular aspects, the cancer is pancreatic cancer. In some aspects, the cancer is lung cancer.

### B. Hexahydrocannabidiol Derivatives

As disclosed herein, certain compounds described herein are shown to be effective in treatment and/or prevention of cancer. Accordingly, in some aspects, the disclosed methods comprise administering a compound of the disclosure to a cancer cell, a subject having cancer, a subject suspected of having cancer, or a subject at risk for developing cancer.

In some aspects, disclosed is a method for treatment or prevention of cancer comprising administering a compound of formula (I):

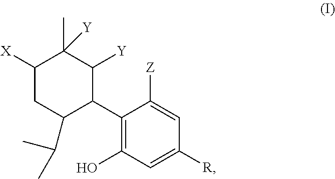

wherein
X is H, halogen, CF₃, or OR²; R² is CF₃, or C₁₋₆ alkyl; Y is H, or two adjacent Y are taken together to form a 3-membered heterocylcoalkyl ring, or two adjacent Y are taken together to form a 3-membered cycloalkyl ring optionally substituted with two R³; each R³ is independently H or F; Z is OR⁴; R⁴ is H, C₁₋₉ substituted or unsubstituted alkyl, C₁₋₉ substituted or unsubstituted alkenyl, C₁₋₉ substituted or unsubstituted alkynyl, OC(O)(CH₂)ₙC(O)—O-gemcitabine, or OC(O)(CH═CH)ₙC(O)—O-gemcitabine, wherein n is an integer from 1-20; R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁵; and R⁵ is H, C₁₋₉ substituted or unsubstituted alkyl, C₁₋₉ substituted or unsubstituted alkenyl, or C₁₋₉ substituted or unsubstituted alkynyl.

In certain aspects, the disclosed compound nomenclature corresponds with the dibenzopan nomenclature, including as shown for carbon 7-10 in formula (I′)

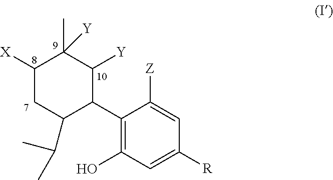

In particular aspects, the compound is H4CBD. In some aspects, the compound is a H4CBD derivative.

Also disclosed are methods comprising administering a compound of formula (I) in combination with one or more chemotherapeutics. In some aspects, the chemotherapeutic is covalently linked to the compound, for example via a linker such as an ester or amino acid linker.

### C. Chemotherapy

In some aspects, the cancer therapy to be administered to the subject comprises a chemotherapy. In some aspects, the chemotherapy is administered in combination with one or more of the other compositions disclosed herein, such as any of the cannabinoids and/or cannabinoid derivatives. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some aspects, gemcitabine, 5-fluorouracil, paclitaxel, nab-paclitaxel, and/or sotorasib is administered in combination with one or more other compounds disclosed herein, such as any cannabinoid and/or cannabinoid derivative described herein. In some aspects, gemcitabine, 5-fluorouracil, paclitaxel, nab-paclitaxel, and/or sotorasib is administered with CCL-104 and/or CCL-106. In some aspects, cisplatin is a particularly suitable chemotherapeutic agent.

In some aspects, gemcitabine, 5-fluorouracil, paclitaxel, nab-paclitaxel, and/or sotorasib are formulated in a pharmaceutical composition in combination with any of the cannabinoid and/or cannabinoid derivatives described herein. In some aspects, the pharmaceutical composition is used in a method to treat a patient, including any method described herein.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain aspects. In some aspects, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”), docetaxel, and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains. Additionally, poly(ADP)-ribose polymerase inhibitors Rucaparib, Olaparib, Veliparib or Talazoparib may be administered using a therapeutic dosage.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### General Pharmaceutical Compositions

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. In some embodiments, the surfactant comprises tween, polysorbate, saponin, tocofersolan (TPGS), lecithin (including sunflower or soy lecithin), sodium stearoyl lactylate, or a combination thereof. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of 5-Butyl-2-(2-Isopropyl-5-Methylcyclohexyl)Benzene-1,3-Diol

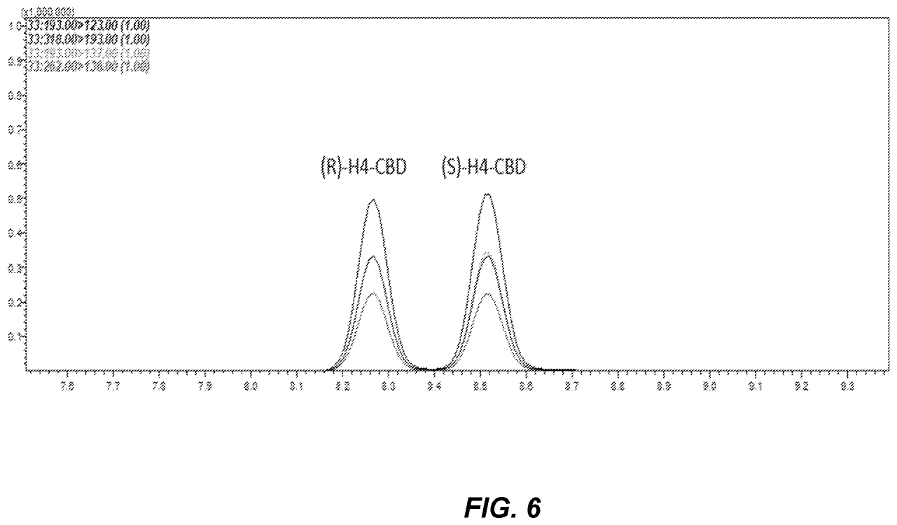

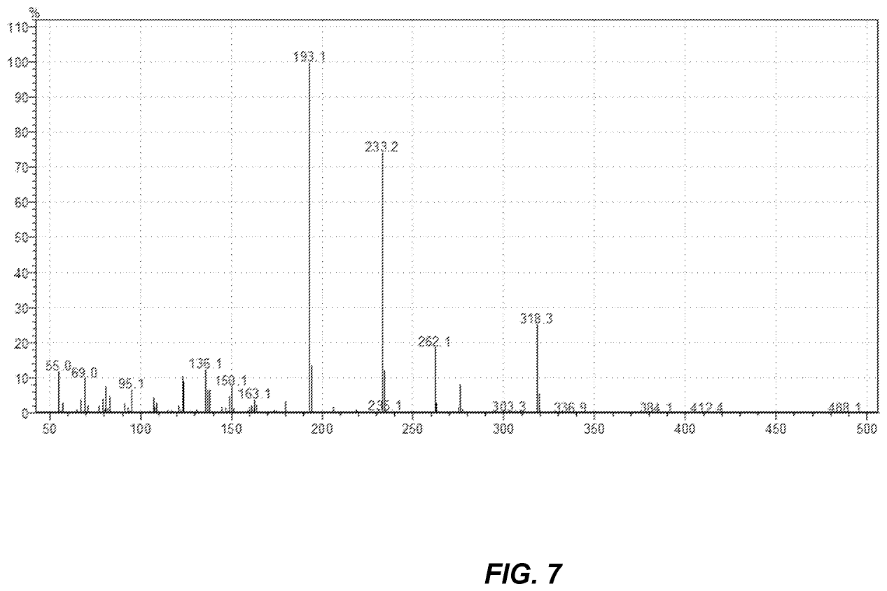

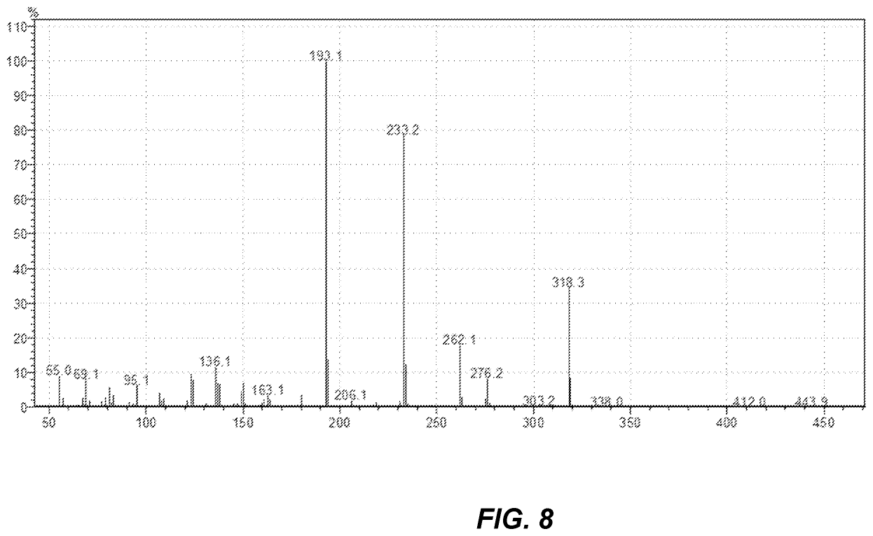

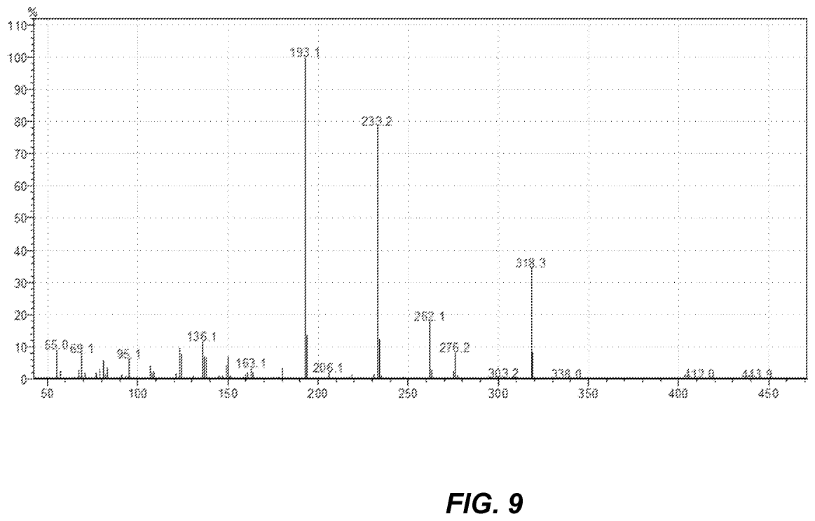

H₄CBD (5-butyl-2-(2-isopropyl-5-methylcyclohexyl)benzene-1,3-diol) was synthesized using a hydrogenation reaction, and a racemic mixture was utilized for a portion of these studies. The racemic mixture was also separated using scCO2 and the studies were repeated using (R)-H4CBD and (S)-H4CBD. HPLC (C18): 4.610, 4.697 min, HRMS (EI): [M*]−318, 1H NMR (500 MHz, CD3CN) δ 6.07 (2H), 6.03 (2H), 3.19 (1H), 2.28 (2H), 2.11 (2H), 1.48 (4H), 1.34 (2H), 1.20 (6H), 0.92 (3H), 0.78 (3H), 0.71 (3H), 0.61 (3H) 13C NMR (500 MHz, CD3CN) δ: 157.4, 156.2, 143.7, 111.6, 110.3, 108.7, 77.8, 50.6, 40.1, 36.7, 36.4, 34.0, 32.7, 32.0, 29.1, 28.5, 26.0, 23.6, 23.3, 19.7, 14.7. HPLC and mass spectrometry results shown in FIGS. 6-9.

### Example 2

### Cytotoxicity of H4CBD and Other Compounds

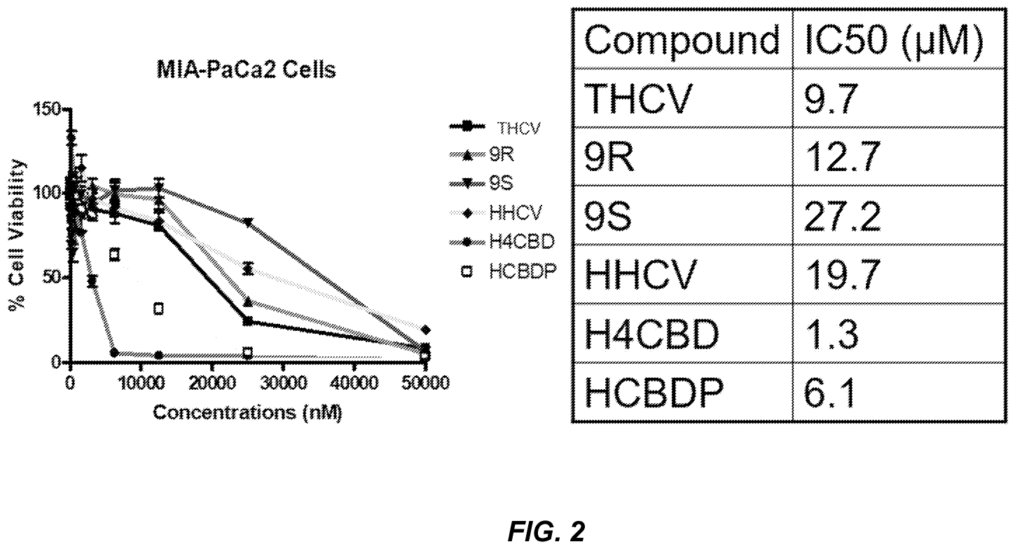

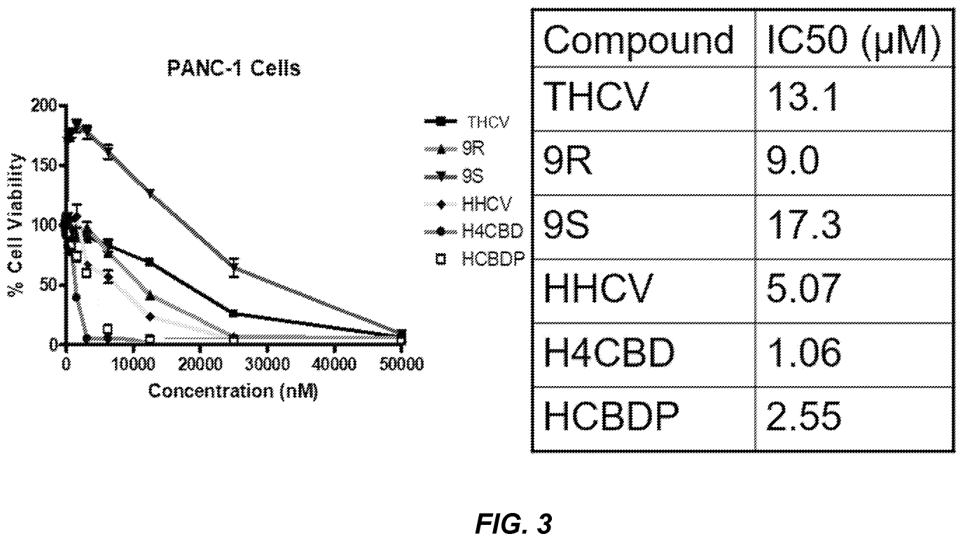

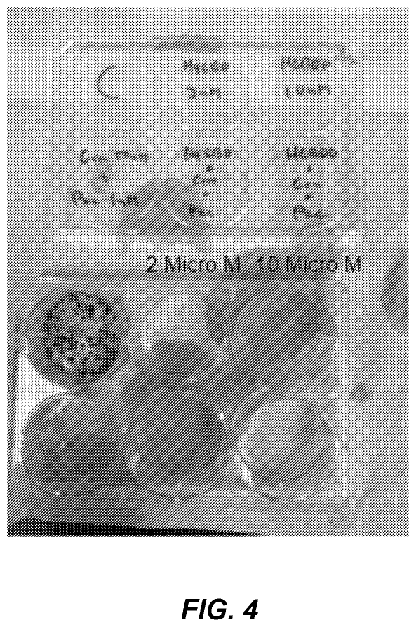

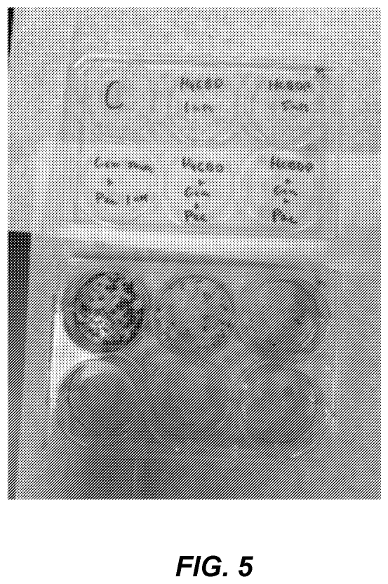

Various compounds, including (R)-H4CBD and (S)-H4CBD, and H4CBDV racemic mixture, were tested for cell killing against various cancer cell lines, including PANC-1, HPAF-II, AsPC-1, and MIA-PaCa2. H4CBDV shows cytotoxicity against all cancer cells tested. Certain results of these studies are shown in FIGS. 2-5.

The IC₅₀ values for (R)-H4CBD, (S)-H4CBD, H4CBDV racemic mixture, and bortezomib were determined. Compound treatment of cells started one day after seeding with a final DMSO concentration of 0.1% and was performed by nanodrop-dispensing using a Tecan Dispenser. 0.1% DMSO (solvent) and Staurosporine (1,0E-05M) served as high control (100% viability) and low control (0% viability), respectively. For the assays, cells were seeded in white cell culture-treated flat and clear bottom multiwell plates and incubated at 37° C. overnight before compounds were added. After incubation for 72 h at 37° C. at 5% or 10% CO2 dependent on the medium, cell plates were equilibrated to room temperature for one hour, CellTiterGlo reagent (Promega) was added and luminescence was measured approximately an hour later using a luminometer.

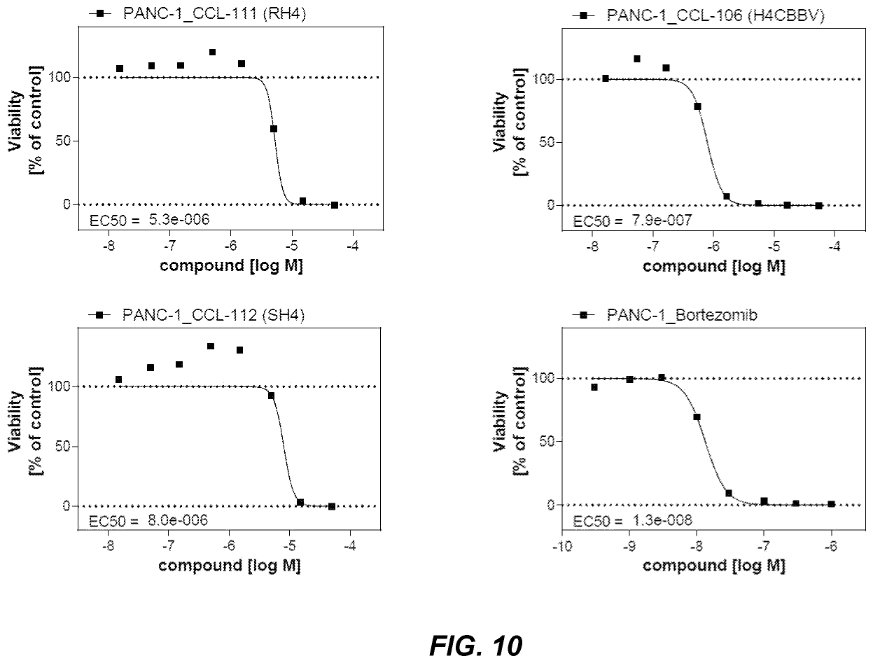

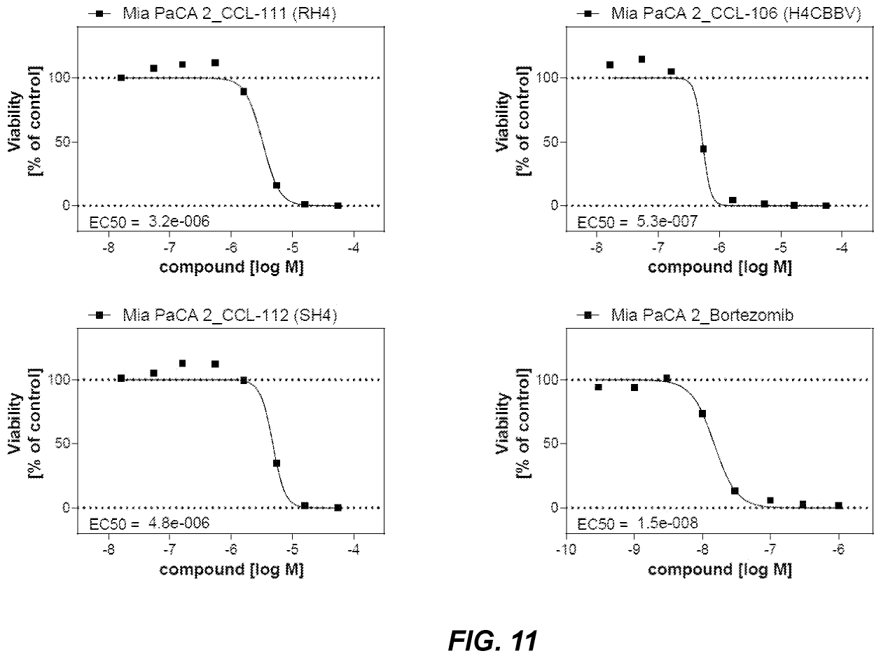

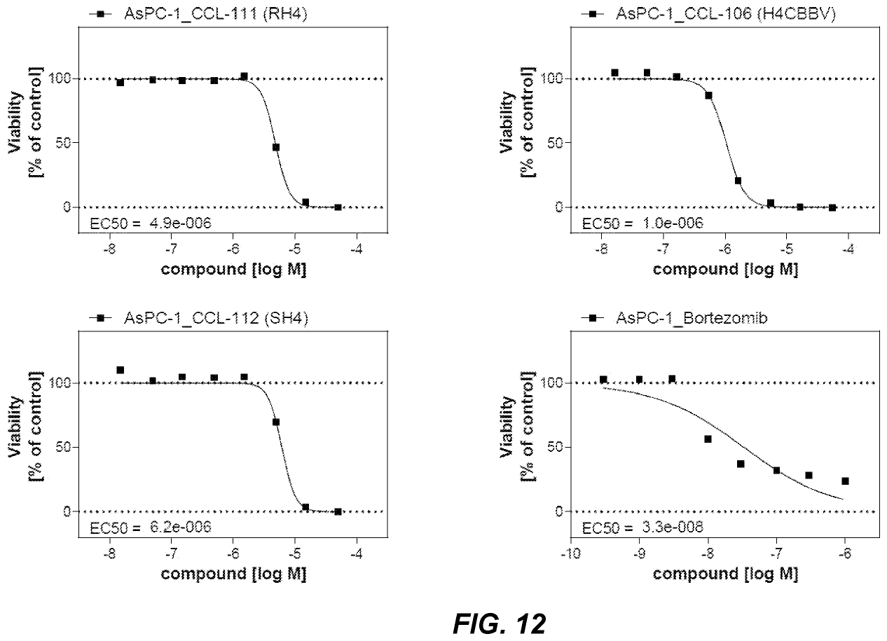

Raw data were converted into percent cell viability relative to the high and low control, which were set to 100% and 0%, respectively. IC₅₀ calculation was performed using GraphPad Prism software with a variable slope sigmoidal response fitting model using 0% viability as bottom constraint and 100% viability as top constraint. IC₅₀ results in PANC-1, MIA-PaCa2, and AsPC-1 cells for (R)-H4CBD, (S)-H4CBD, H4CBDV racemic mixture, and bortezomib are shown in FIGS. 10-12 and Table 2

TABLE 2
IC₅₀ values in Pancreatic Cancer Cell Lines

| | | IC50 [M] | IC50 [M] | IC50 [M] |
|---|---|---|---|---|
| No. | Compound | AsPC-1 | MiaPaCA-2 | PANC-1 |
| 1 | (R)-H4CBD | 4.85E−06 | 3.24E−06 | 5.29E−06 |
| 2 | H4CBD V | 1.05E−06 | 5.30E−07 | 7.89E−07 |
| | (racemic) | | | |
| 3 | (S)-H4CBD | 6.17E−06 | 4.77E−06 | 8.02E−06 |
| 4 | Bortezomib | 3.34E−08 | 1.49E−08 | 1.34E−08 |

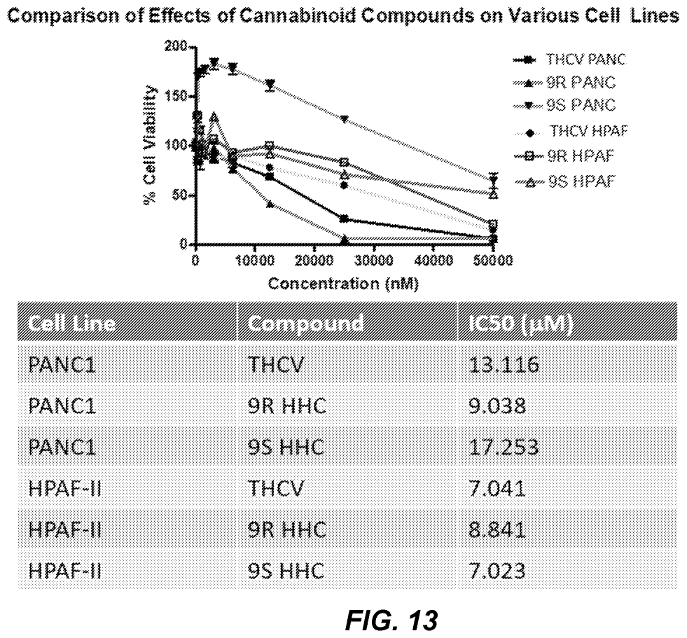

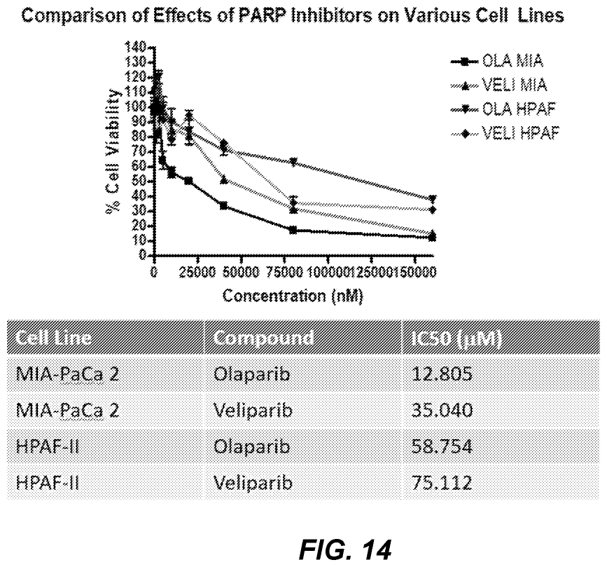

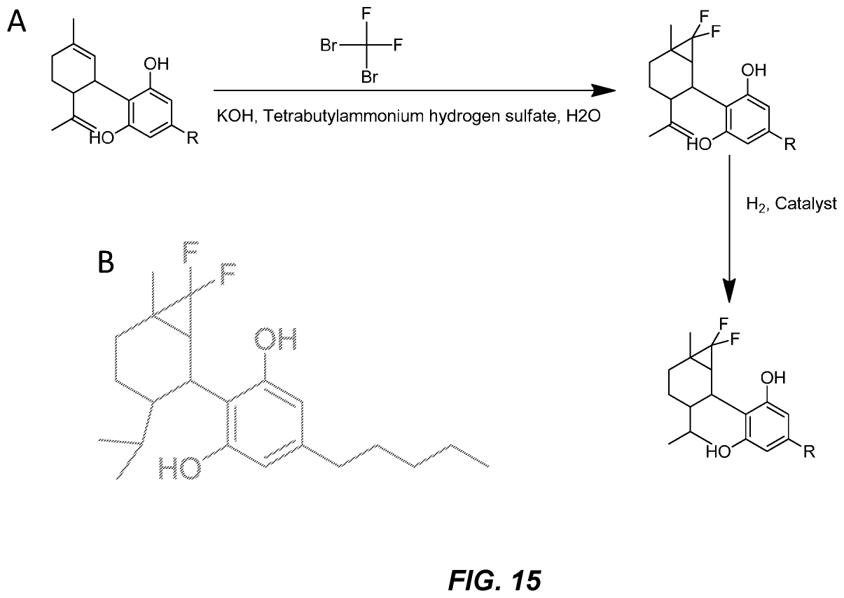

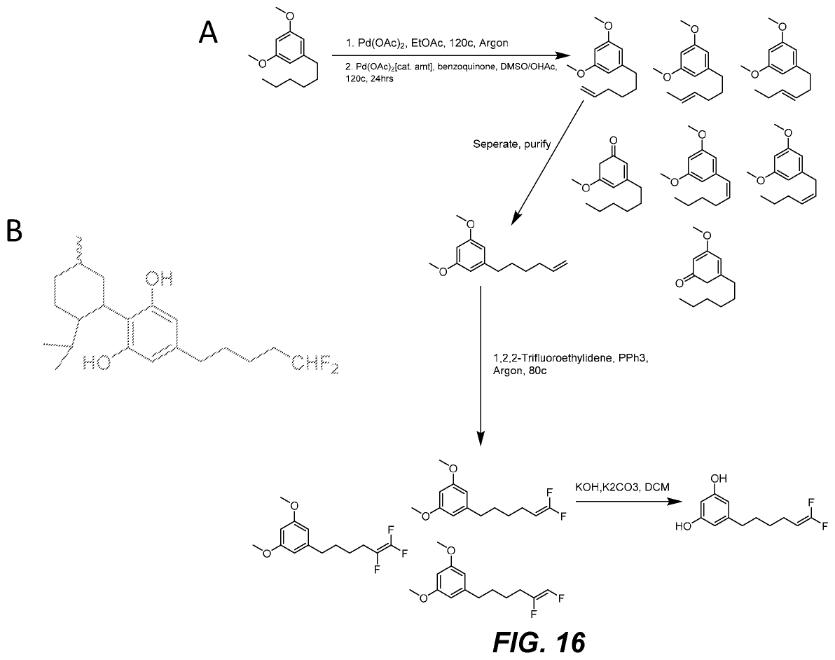

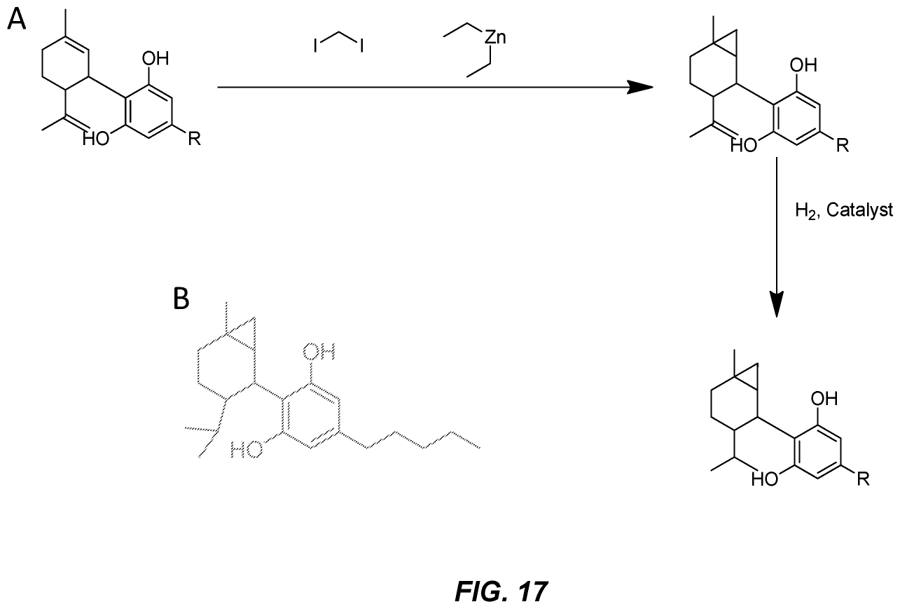

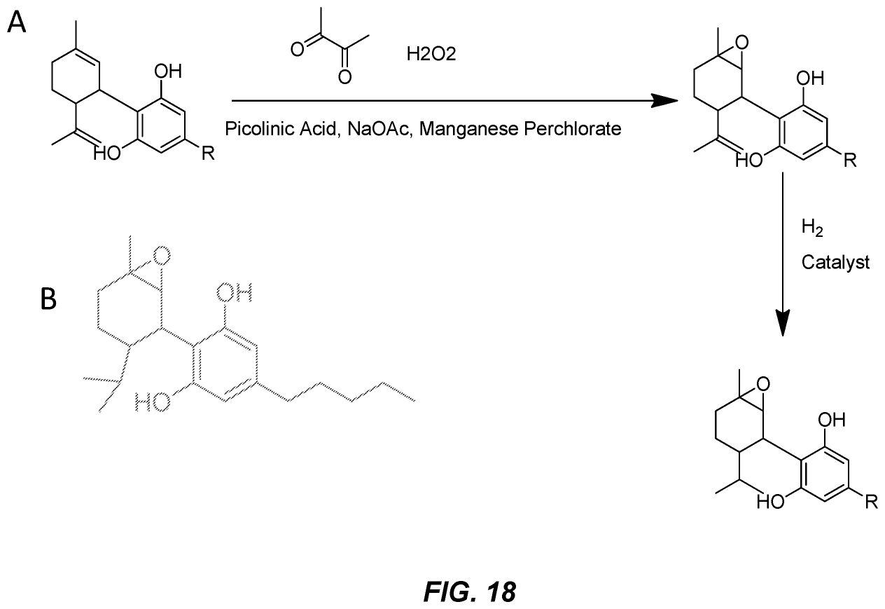

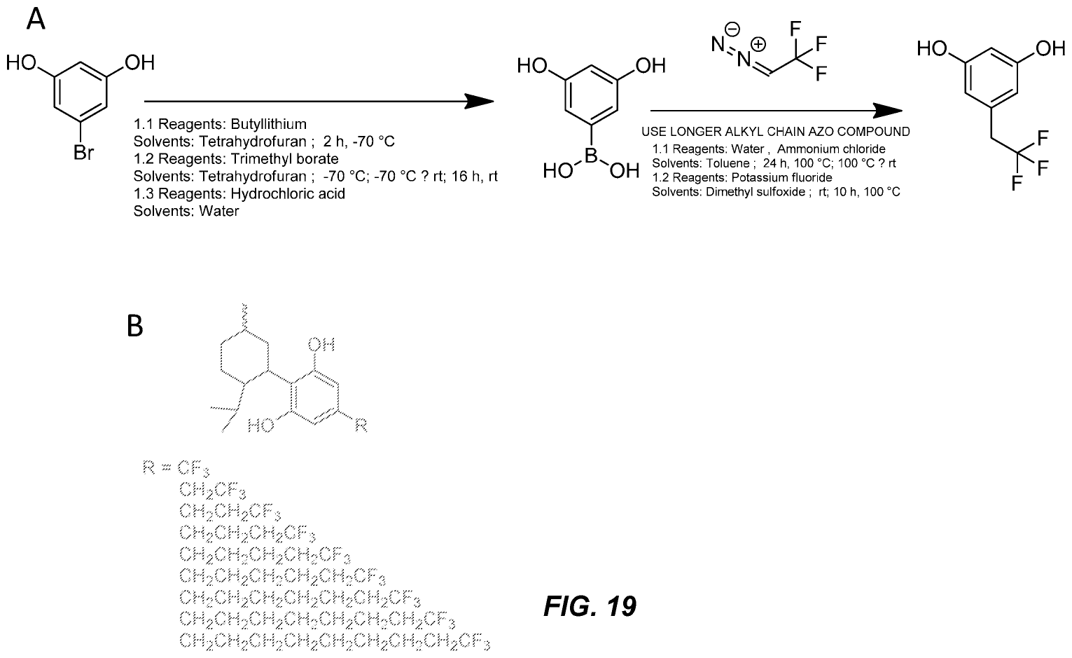

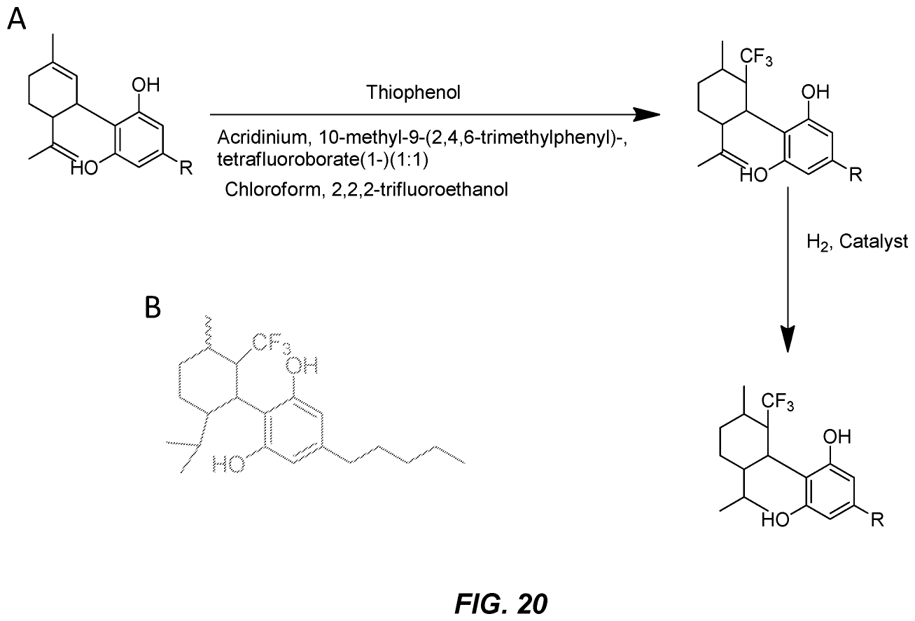

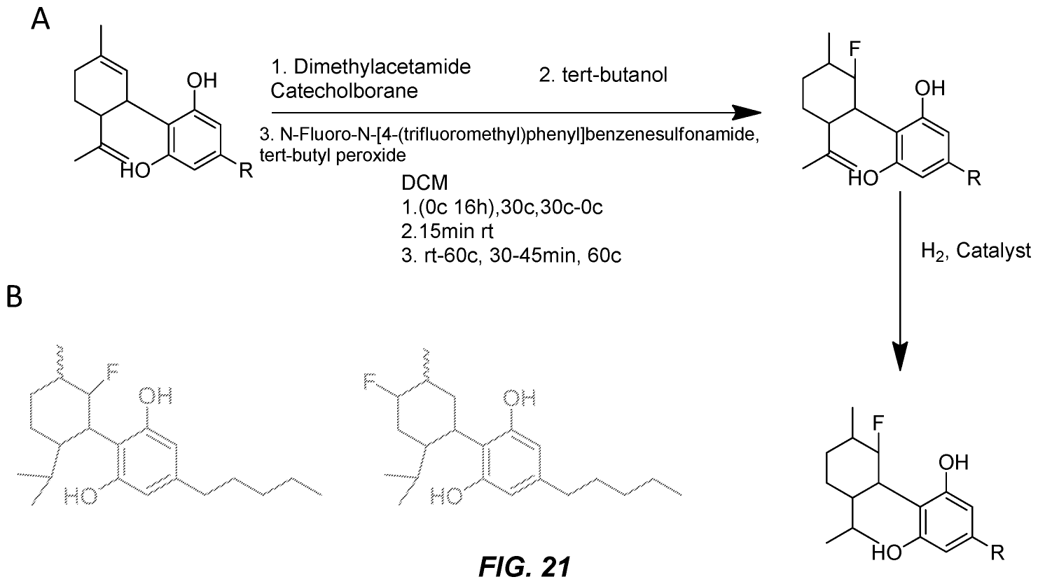

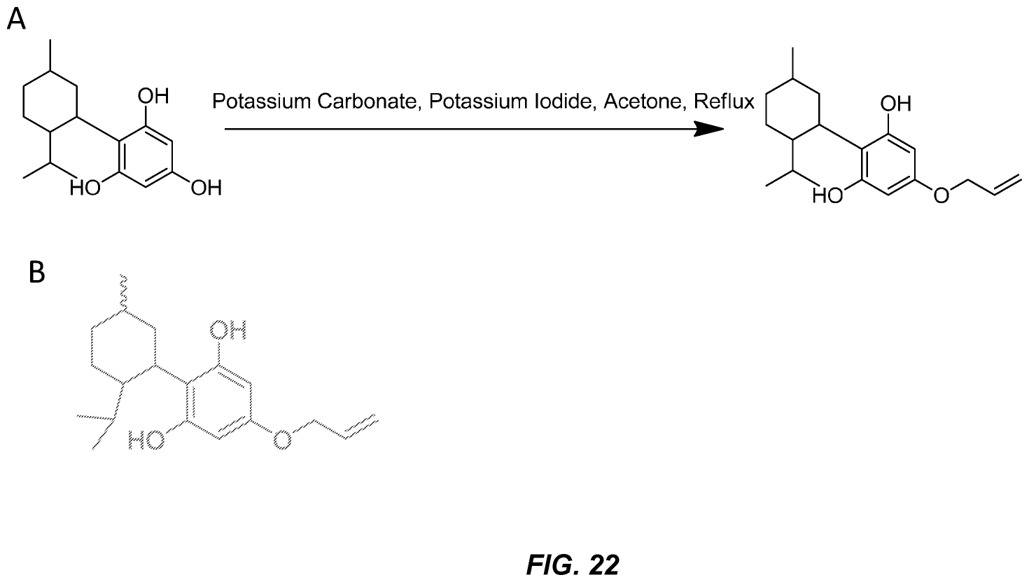

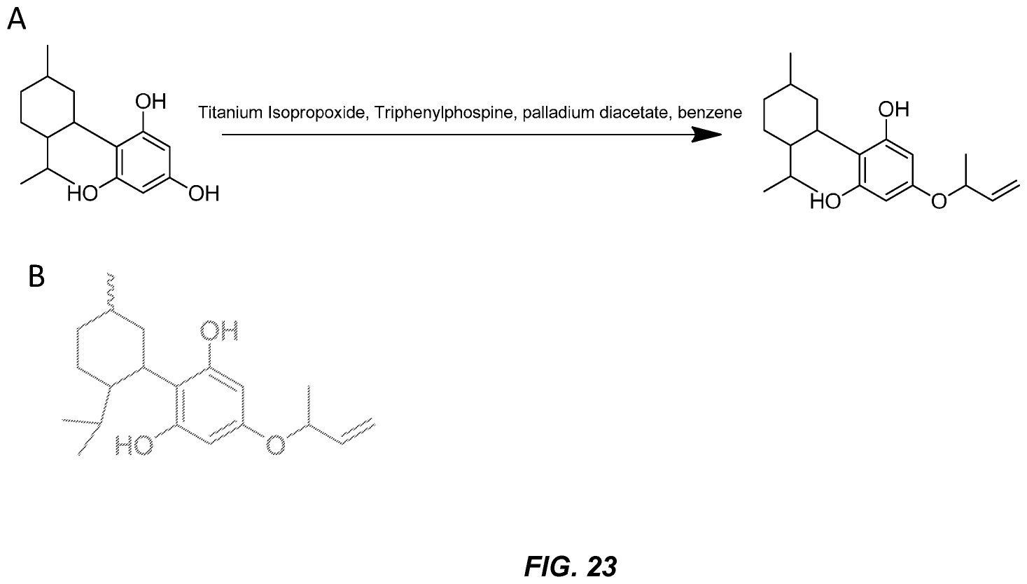

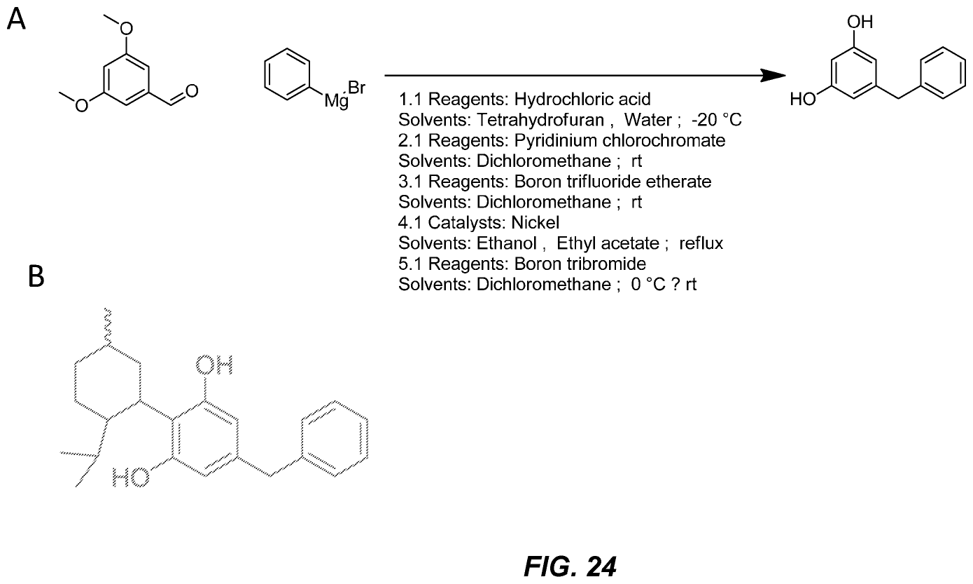

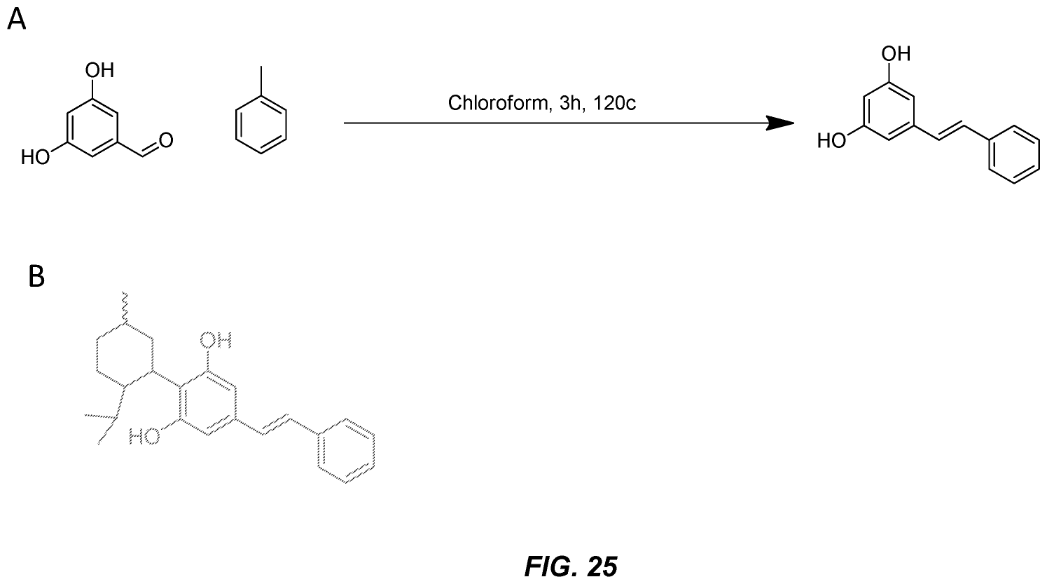

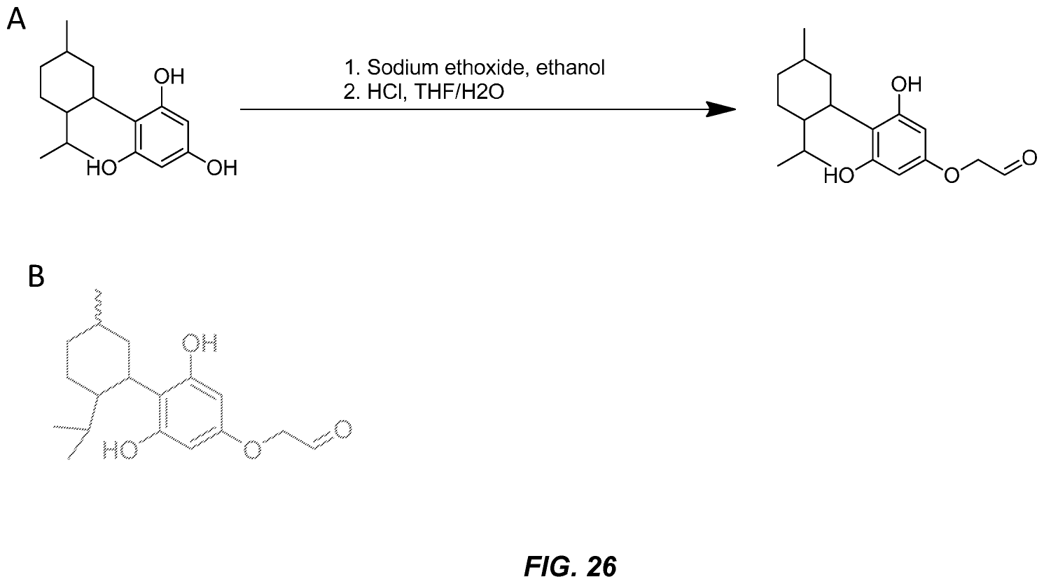

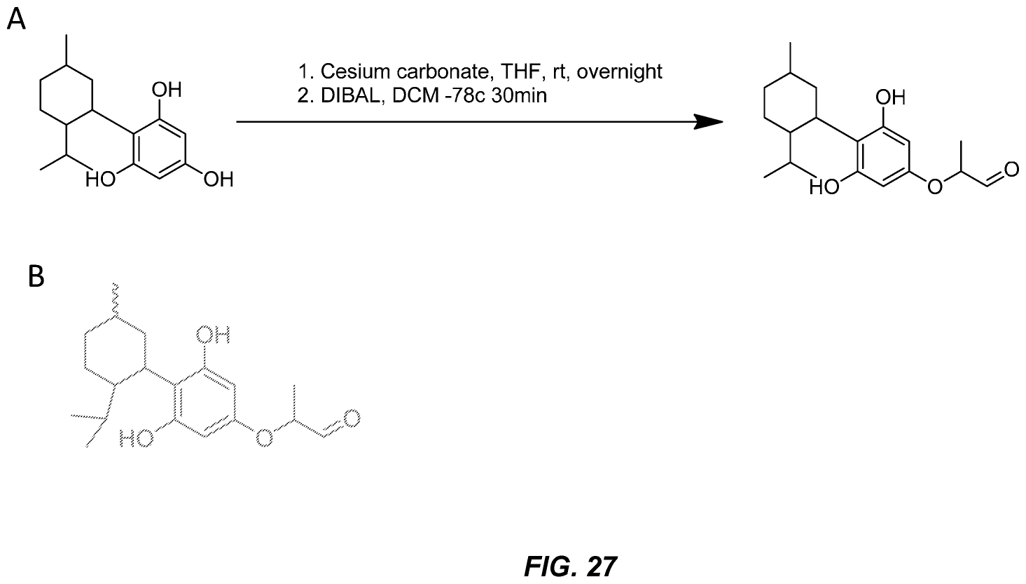

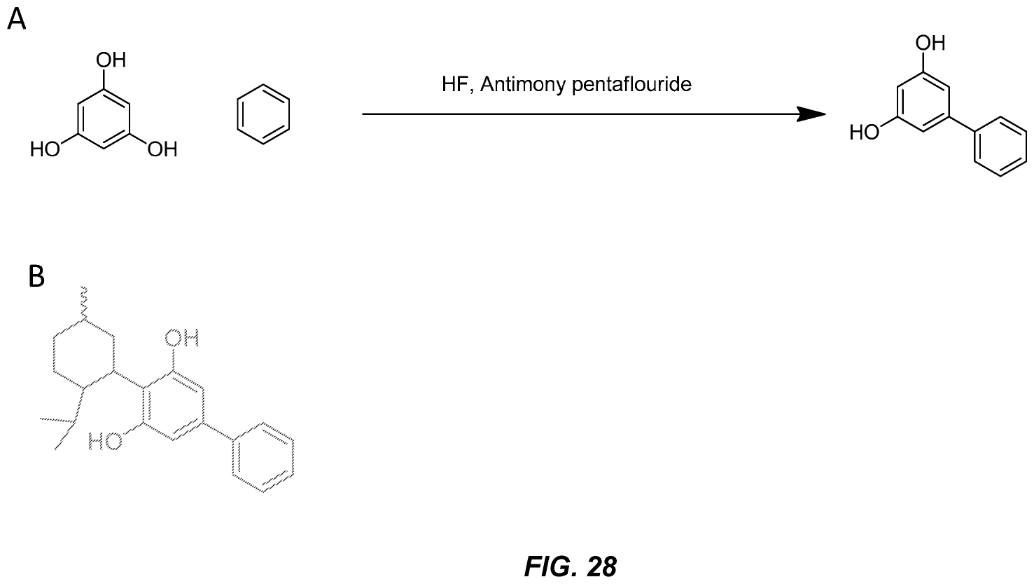

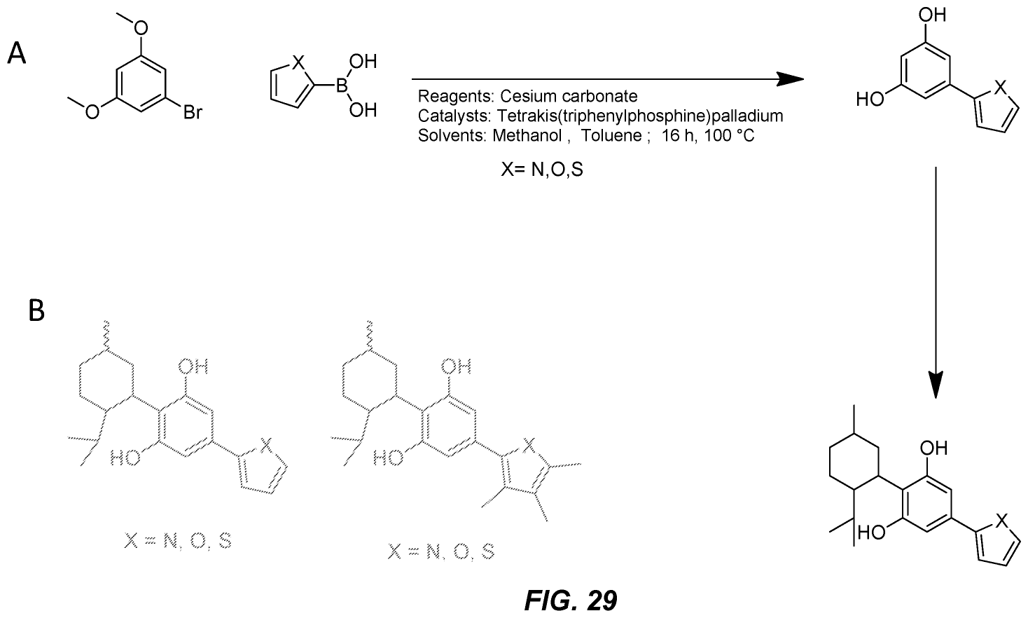

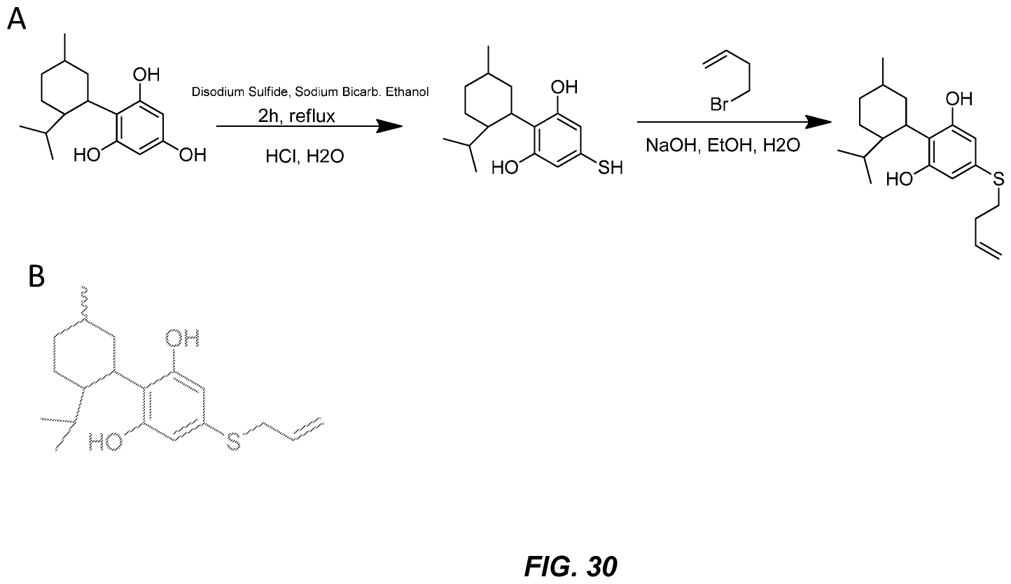

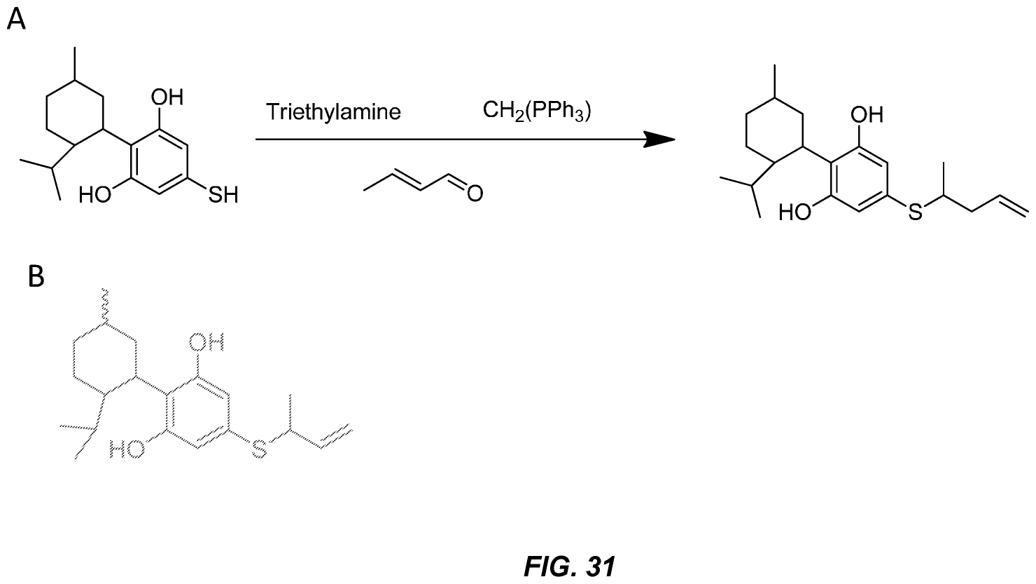

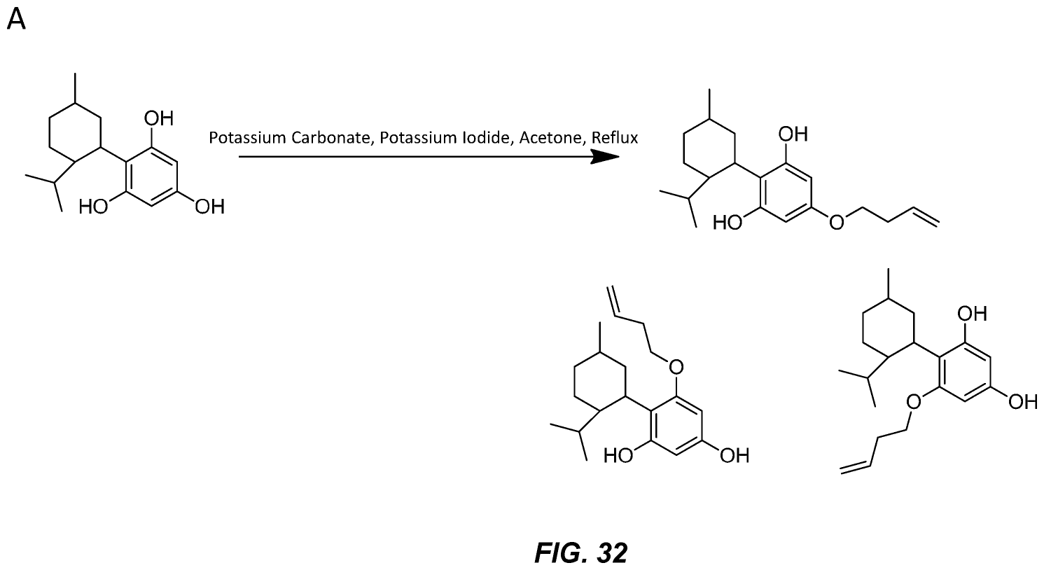

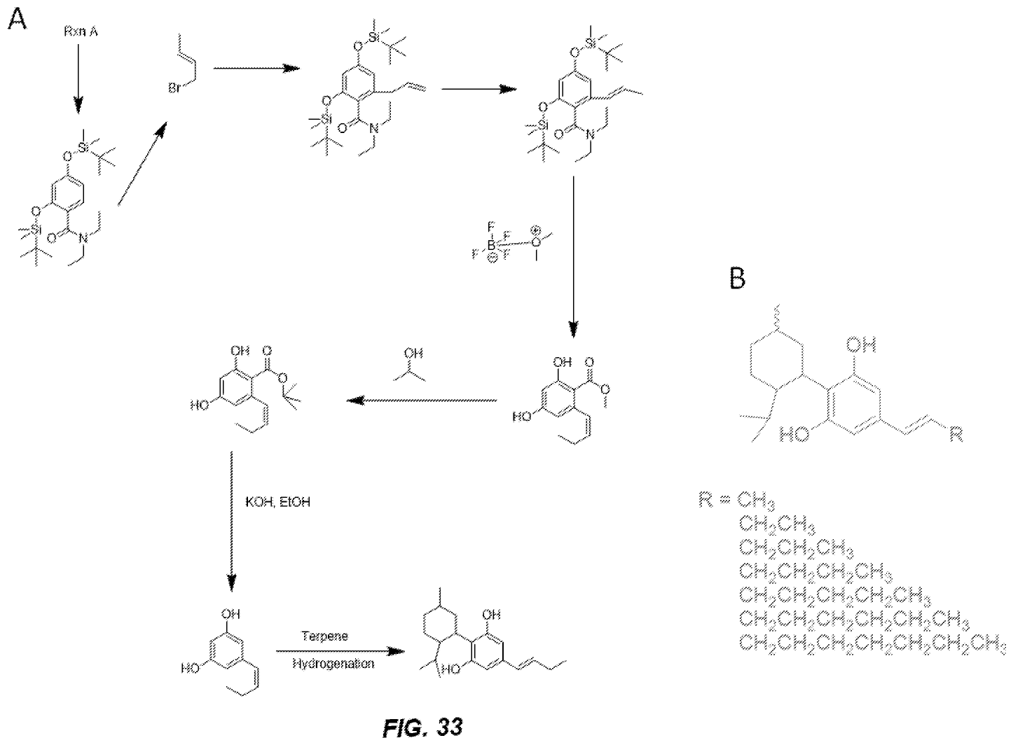

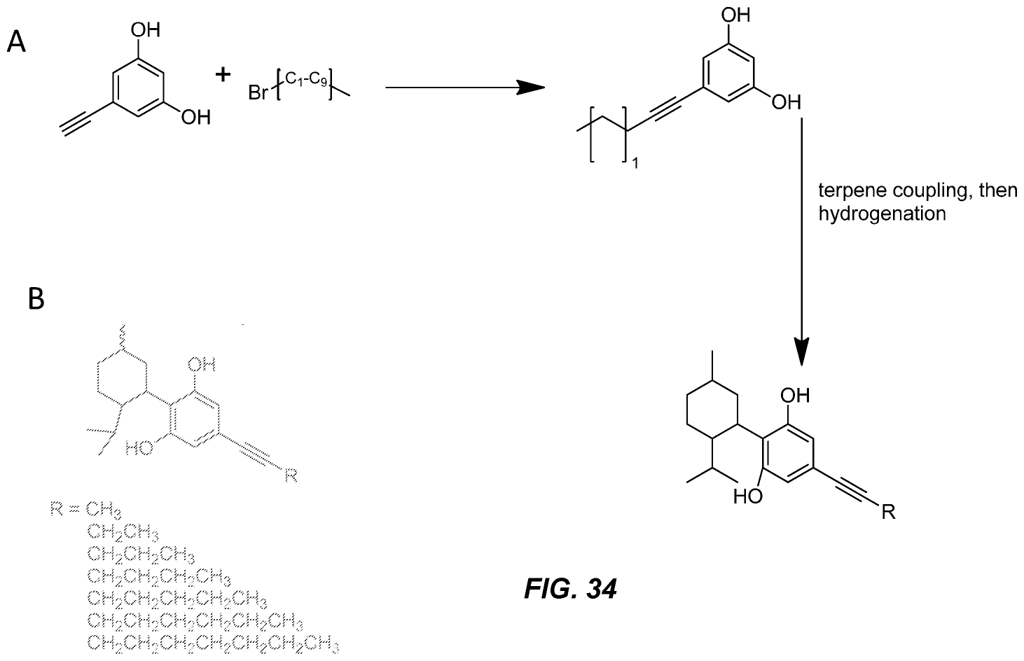

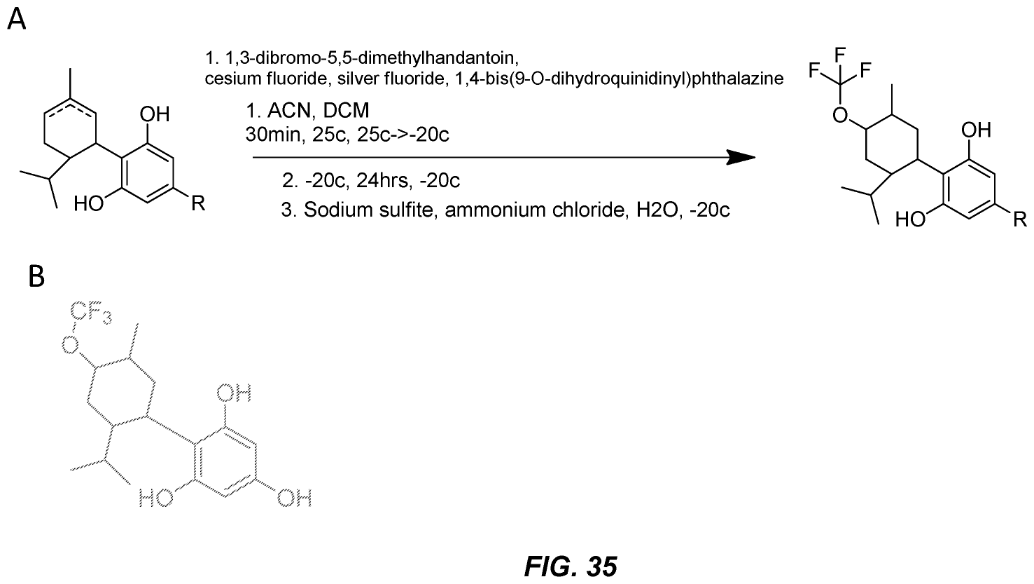

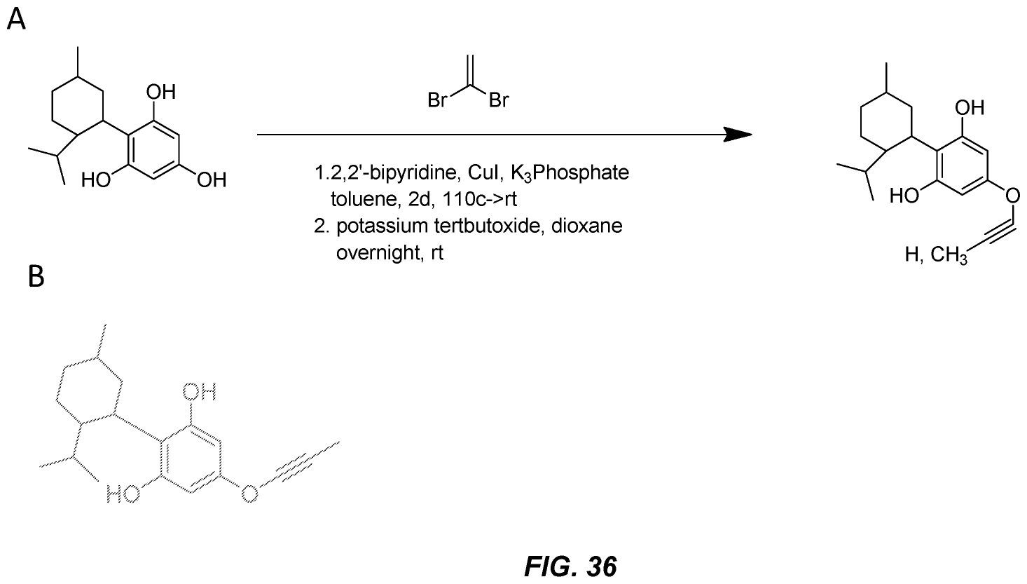

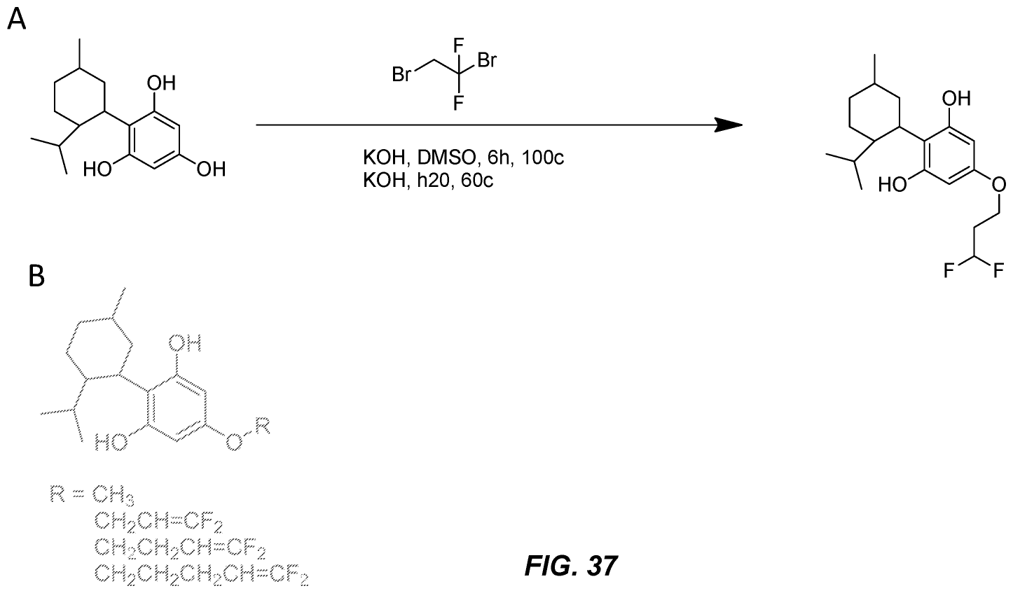

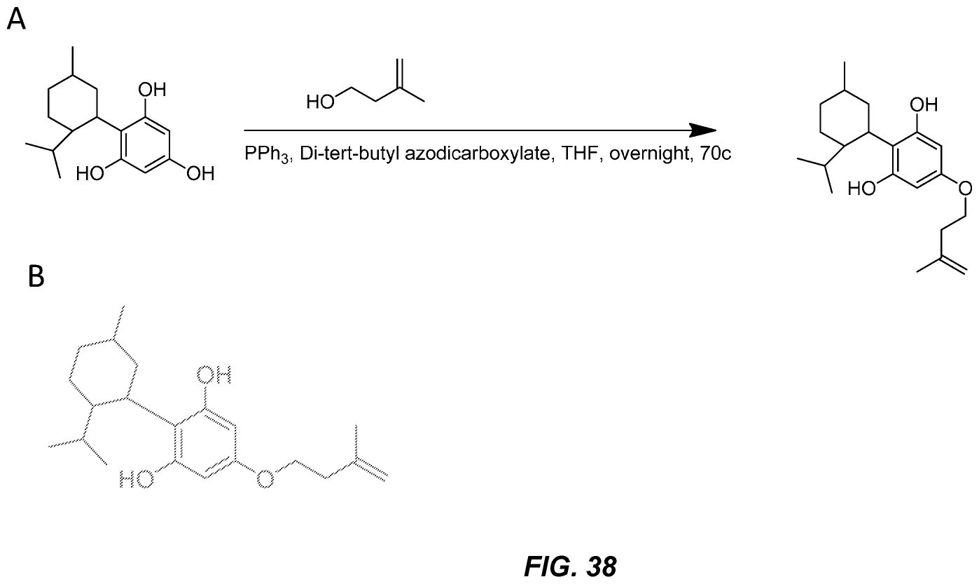

The IC₅₀ of THCV and HHC on PANC-1 and HPAF-II cells were also tested as shown in FIG. 13. The IC₅₀ of Olaparib and Veliparib on PANC-1 and HPAF-II cells were also tested for comparison as shown in FIG. 14.

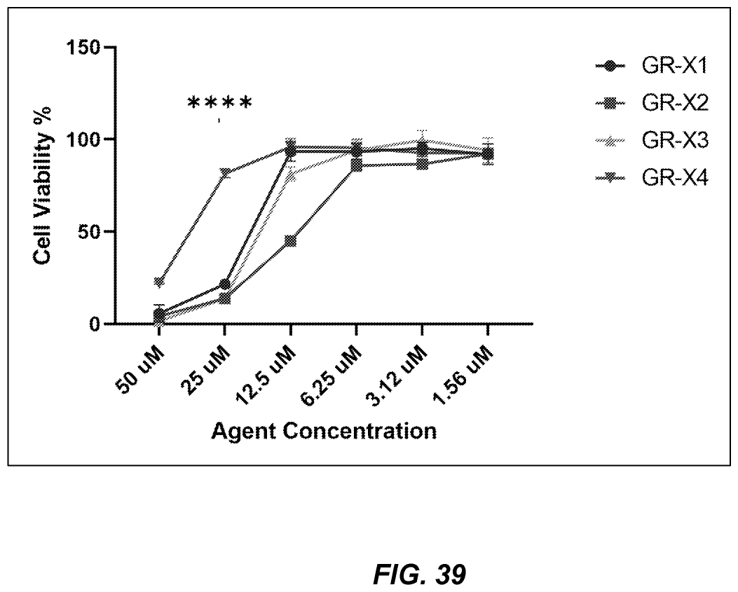

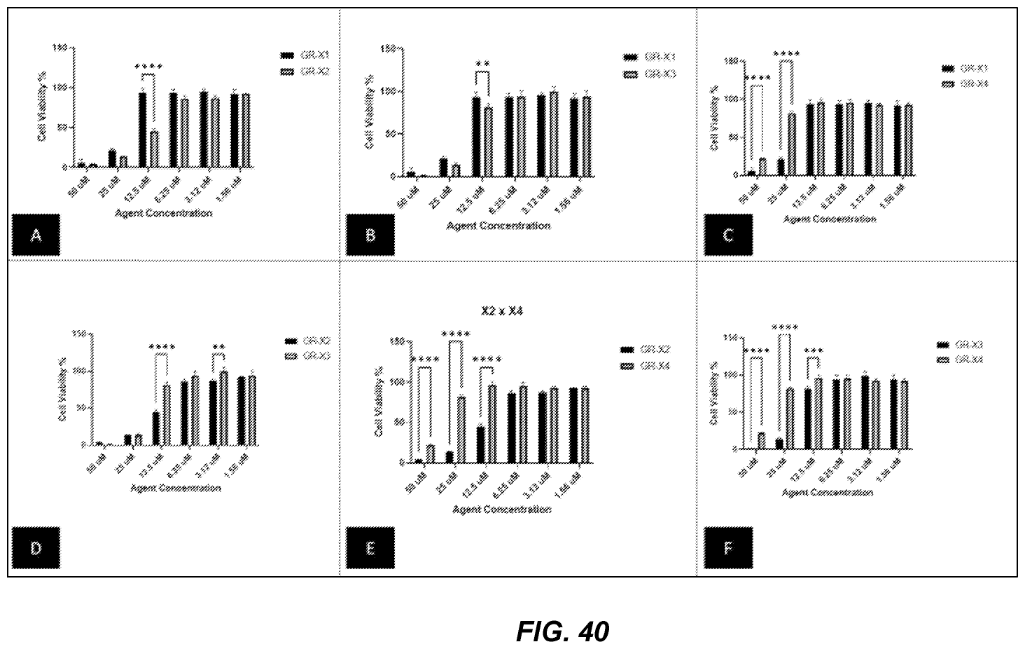

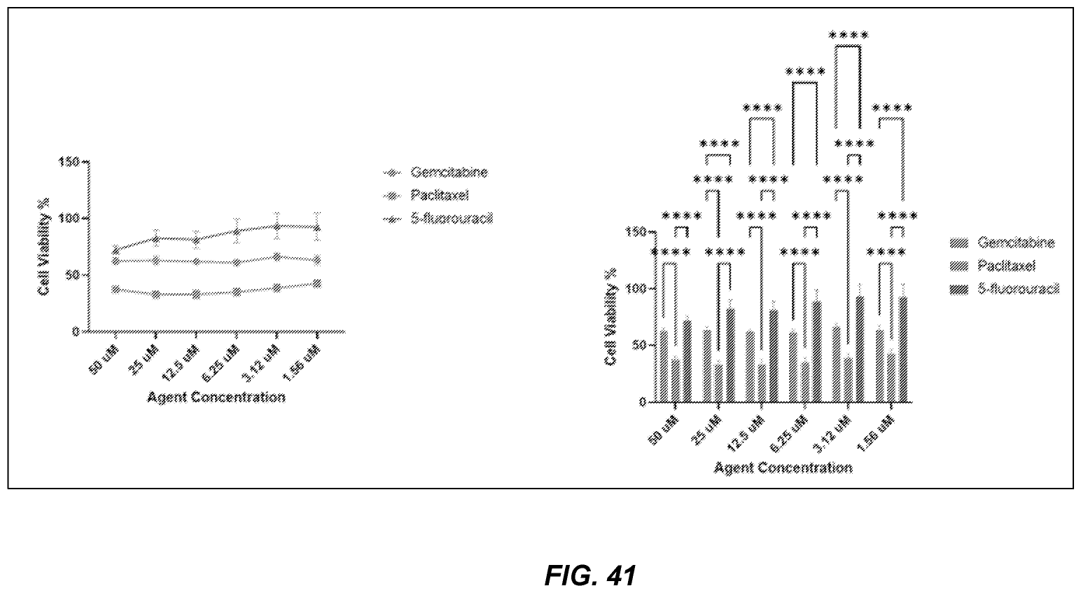

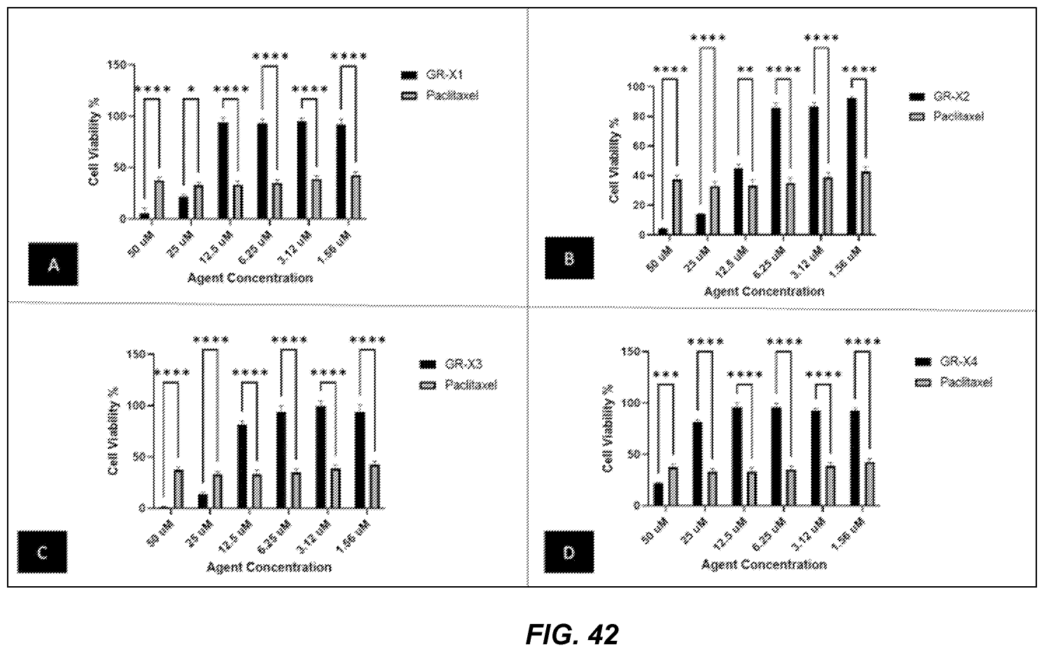

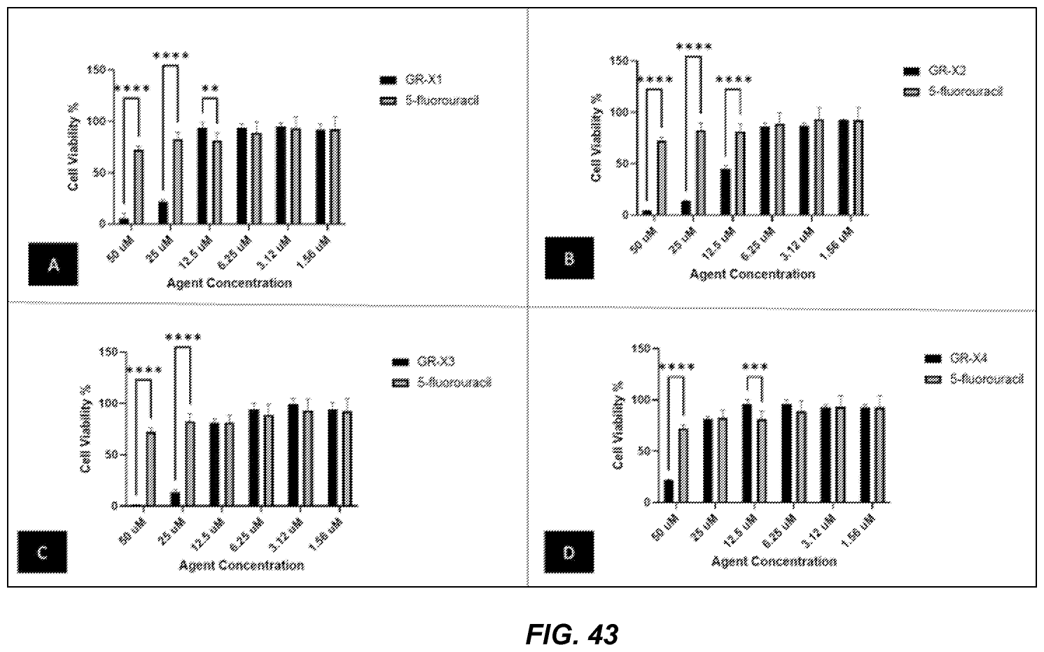

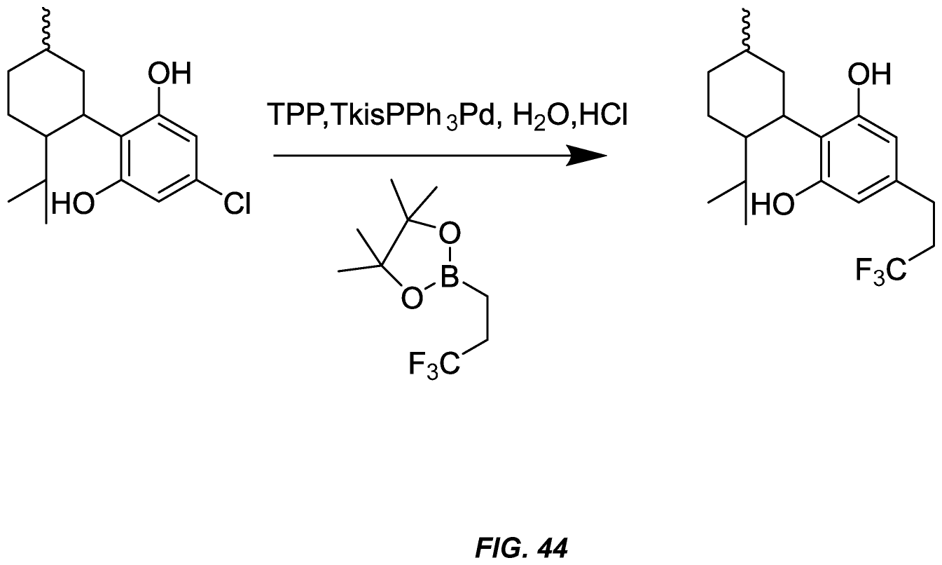

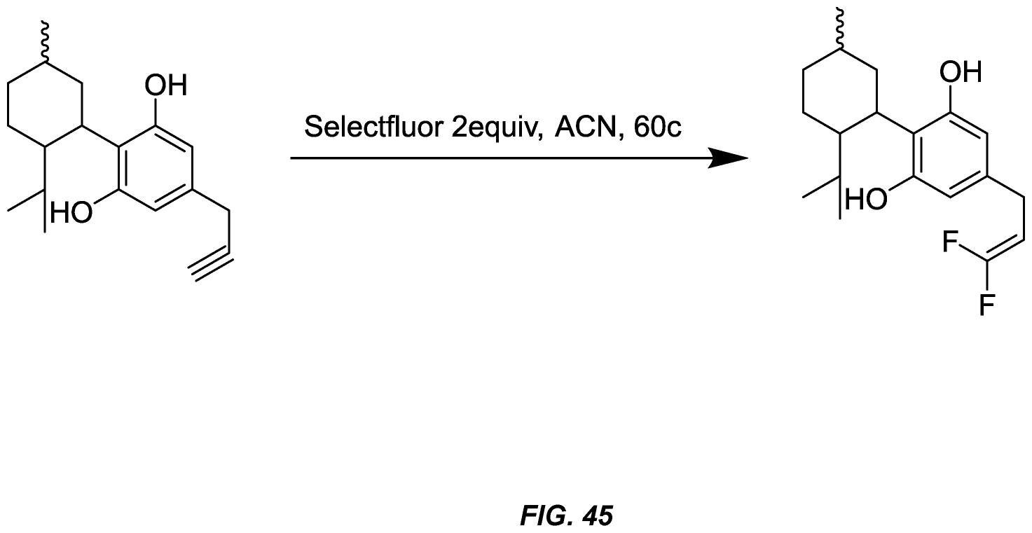

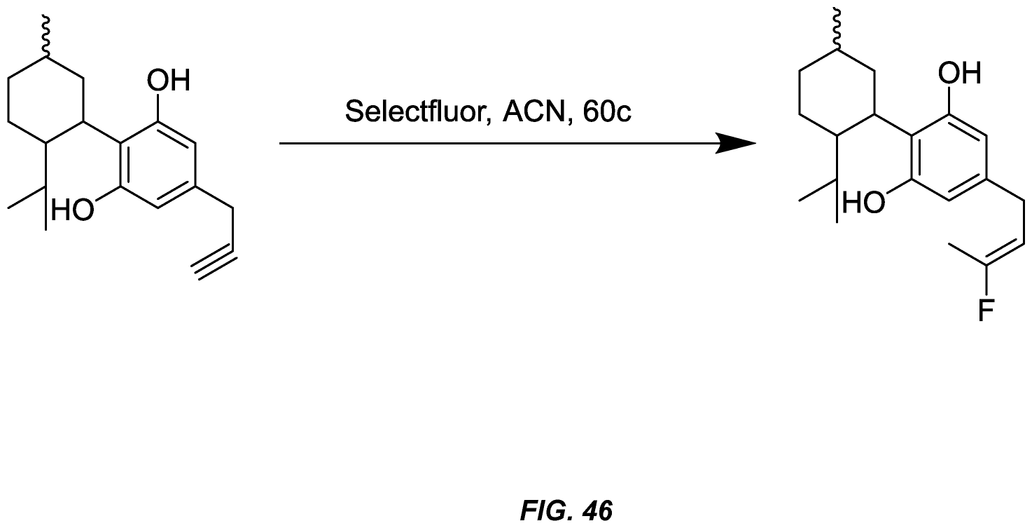

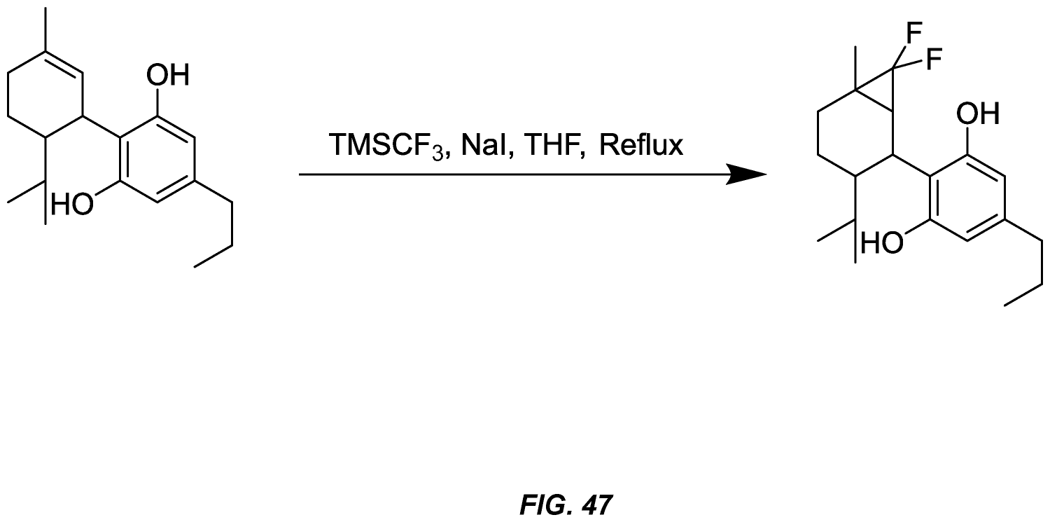

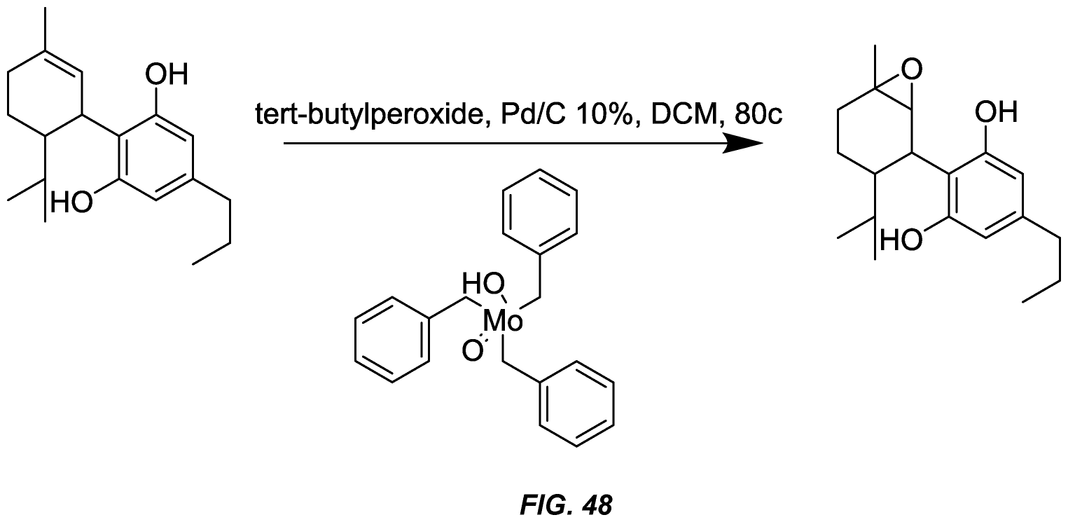

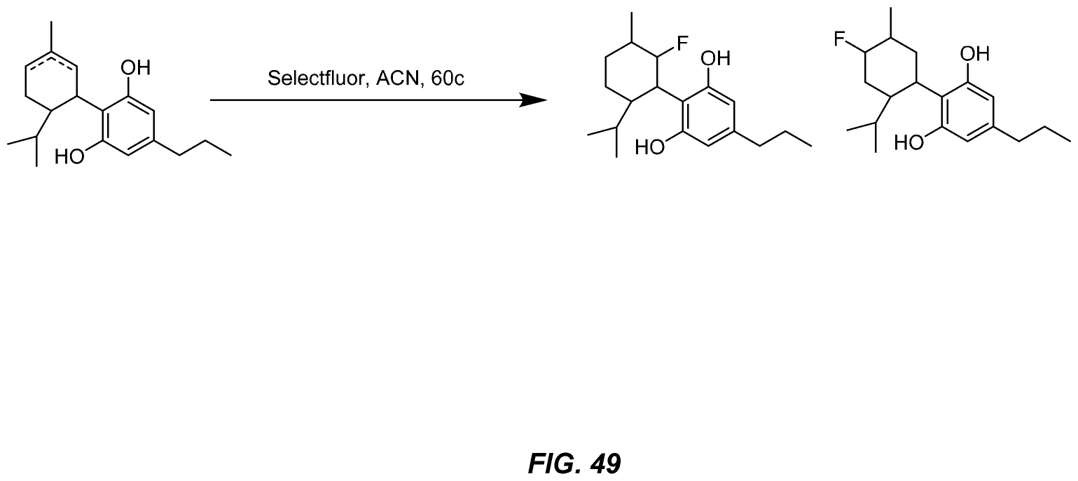

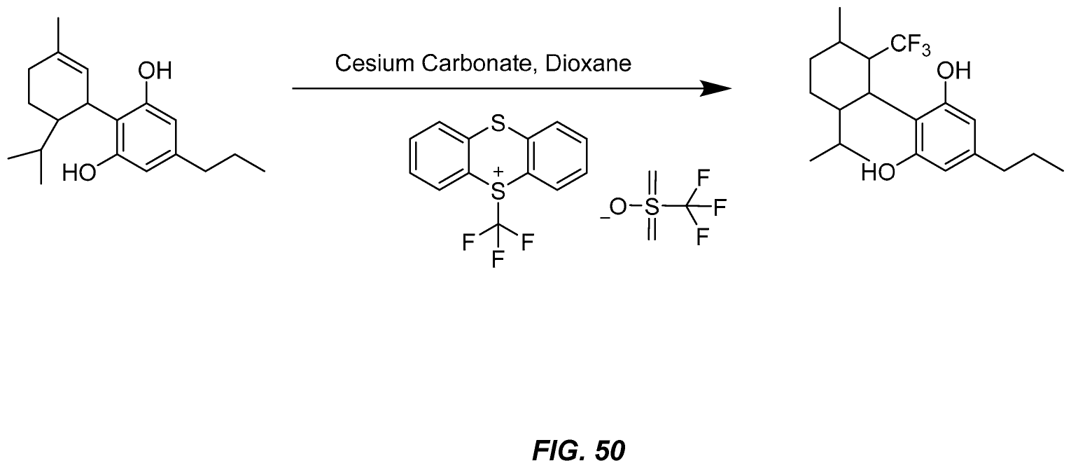

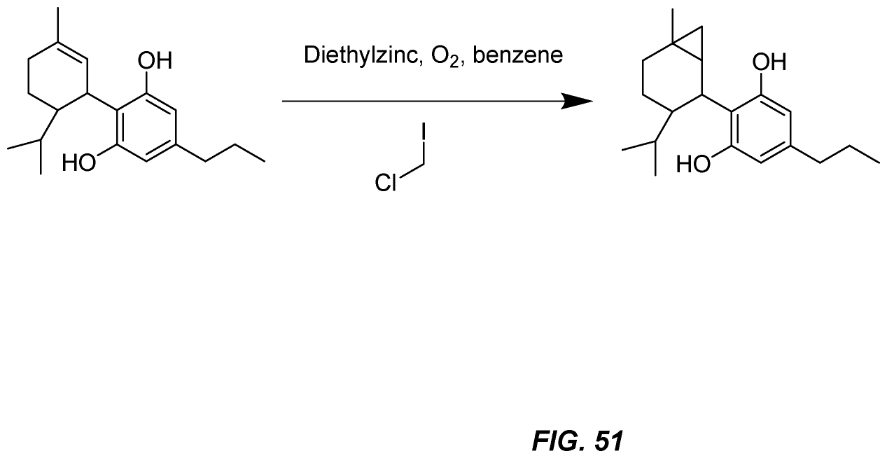

The IC₅₀ of the hydrogenated cannabivarin, hexahydrocannabidivarin (H4CBDV), (R)-H4CBD, (S)-H4CBD, HHC racemic mixture, and gemcitabine, paclitaxel, and 5-fluorouracil were determined in lung carcinoma cells (A549 Lung Carcinoma Epithelial Cells (CCL-185 from ATCC)) as shown in FIGS. 39-43 and Table 3.

TABLE 3
Percent viability of cells treated with cannabinoid derivatives.

| | | | H4CBDV | | R- H4CBDV | | S- H4CBDV | | HHC | |
|---|---|---|---|---|---|---|---|---|---|---|
| | | | Mean | | Mean | | Mean | | Mean | |
| | | | (%) | SD | (%) | SD | (%) | SD | (%) | SD |
| Agent | 50 | uM | 5.65 | 4.09 | 4.18 | 0.34 | 1.53 | 0.10 | 22.07 | 0.48 |
| concentration | 25 | uM | 21.54 | 2.02 | 13.97 | 0.31 | 13.77 | 1.68 | 81.59 | 1.87 |
| | 12.5 | uM | 93.59 | 4.68 | 45.02 | 2.56 | 81.32 | 3.30 | 96.09 | 3.76 |
| | 6.25 | uM | 93.41 | 3.60 | 85.97 | 2.69 | 94.14 | 5.30 | 95.63 | 3.68 |
| | 3.12 | uM | 95.15 | 2.83 | 86.74 | 2.13 | 99.61 | 4.62 | 92.87 | 1.79 |
| | 1.56 | uM | 92.07 | 4.73 | 92.31 | 0.85 | 94.17 | 5.78 | 92.47 | 2.45 |

Table 4 summarizes the measured IC50 for specific compounds in the indicated cell types.

TABLE 4
IC₅₀ values in various cell lines for CCL-100 to CCL-105.

IC50 mM

| Compound | PANC1 | HPAFII | MIA-PaCa2 | AsPC-1 |
|---|---|---|---|---|
| CCL-100 | 13.11 | 7.04 | 9.7 | 6.52 |
| CCL-101 | 9.08 | 8.84 | 12.7 | 16.61 |
| CCL-102 | 17.25 | 7.02 | 27.2 | 11.51 |
| CCL-103 | 5.07 | | 19.7 | |
| CCL-104 | 1.06 | | 1.3 | |
| CCL-105 | 2.55 | | 6.1 | |

CCL-113 did not show cytotoxicity in an Ames test nor in a hERG assay. CCL-113 had an IC₅₀ of 14.4 μM in human fibroblasts. At 50 μM, 74.8% of human fibroblasts died and 8% of hepatocytes.

### Example 3

### Formulation Data

Formulation and dosing information for pharmacokinetic data is provided in Table 5 (CD=beta-cyclodextrin). The solutions were sonicated, vortexed, and slightly heated resulting in a clear purple liquid.

TABLE 5
Example formulation of compounds

| Study Type | Standard PK |
|---|---|
| N/Treatment | 4 |

| Doses | IV | 10 | mg/kg |
|---|---|---|---|
| | PO | 10 | mg/kg |
| Formulations | IV | 20% HP-β-CD in water | |
| | PO | 20% HP-β-CD in water | |
| Dosing Solutions | IV | 2 | mg/mL |
| | PO | 1 | mg/mL |

### Example 4

### Pharmacokinetics and Pharmacodynamics

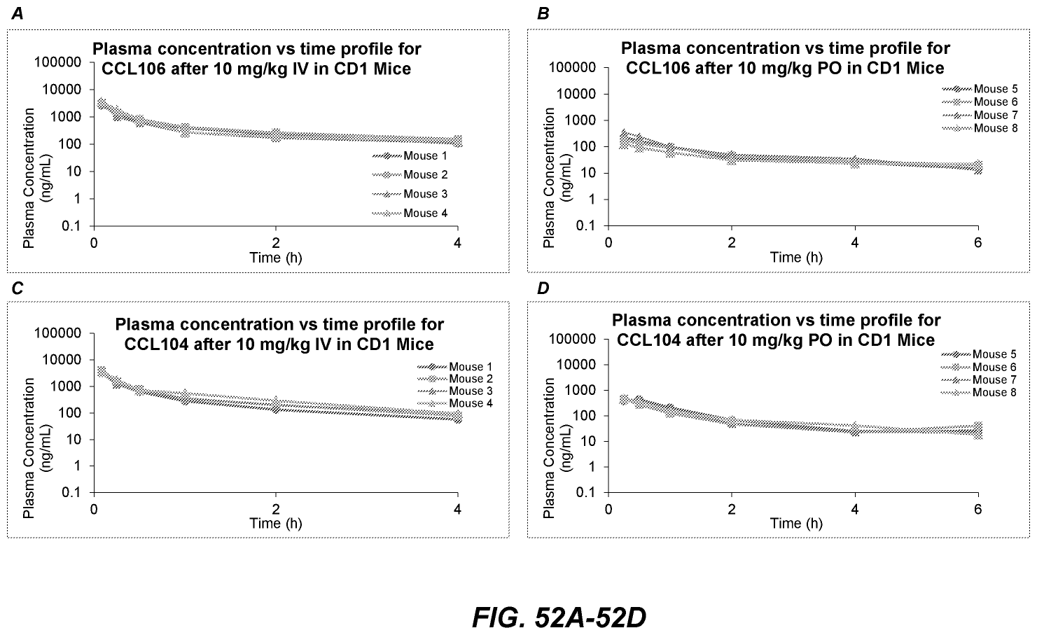

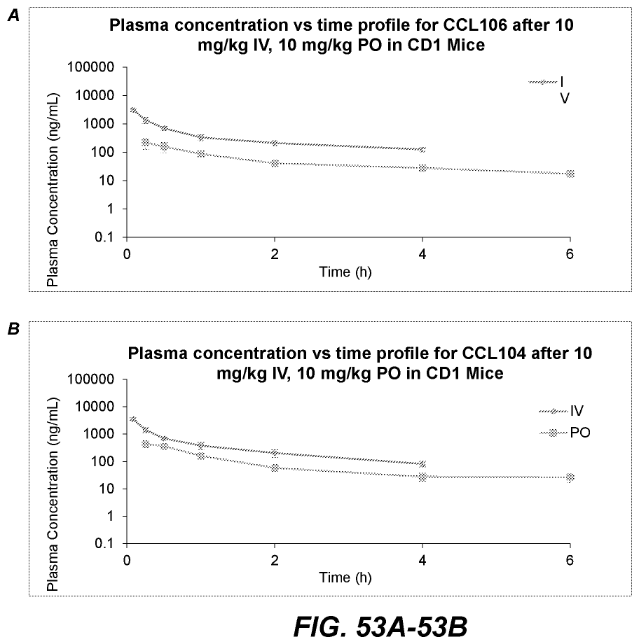

Pharmacokinetic data was produced by administering CD1 male mice with CCL-104 (H4CBD) or CCL-106 (H4CBDV). Each compound was formulated and administered as shown in Example 3. Plasma was sampled from the IV administered mice at 5 min, 15 min, 30 min, 1 hr, 2 hr, 4 hr post dose. Plasma was sampled from the PO administered mice at 15 min, 30 min, 1 hr, 2 hr, 4 hr, 6 hr post dose. Plasma concentrations of each compound were measured in each mouse by HPLC-MS as shown in FIGS. 52A-52D and 53A-53B. The desired serial concentrations of working solutions were achieved by diluting stock solution of analyte with 50% acetonitrile in water solution. 10 μL of working solutions (0.5, 1, 2, 5, 10, 50, 100, 500, 1000 ng/mL) were added to 10 μL of the blank male CD1 mice plasma to achieve calibration standards of 0.5-1000 ng/mL (0.5, 1, 2, 5, 10, 50, 100, 500, 1000 ng/mL) in a total volume of 20 μL. Six quality control samples at 1 ng/mL, 2 ng/mL, 5 ng/mL, 10 ng/mL, 50 ng/mL and 800 ng/mL for plasma were prepared independently of those used for the calibration curves. These QC samples were prepared on the day of analysis in the same way as calibration standards. 20 μL standards, 20 μL QC samples and 20 μL unknown samples (10 μL plasma with 10 μL blank solution) were added to 200 μL of methanol containing IS mixture for precipitating protein respectively. Then the samples were vortexed for 30 seconds, then centrifuged at 4° C., 4,000 rpm for 15 min. The supernatant was diluted with water at a ratio of 1:2. 15 μL of the diluted supernatant was injected into the LC/MS/MS system for quantitative analysis.

A summary of CCL-104 pharmacokinetic parameters when administered IV are shown in Table 6. A summary of CCL-104 pharmacokinetic parameters when administered PO are shown in Table 7. A summary of CCL-106 pharmacokinetic parameters when administered IV are shown in Table 8. A summary of CCL-106 pharmacokinetic parameters when administered PO are shown in Table 9.

TABLE 6
CCL-104 pharmacokinetic parameters when administered IV

| PK parameters | Mouse 1 | 2 | 3 | 4 | Mean | SD | CV(%) |
|---|---|---|---|---|---|---|---|
| Cl_obs | 98.3 | 76.4 | 82.6 | 68.8 | 81.5 | 12.5 | 15.4 |
| (mL/min/kg) | | | | | | | |
| T_{1/2} (h) | 1.35 | 1.42 | 1.63 | 1.19 | 1.40 | 0.19 | 13.3 |
| C₀ (ng/mL) | 5246 | 6608 | 5306 | 5264 | 5606 | 668 | 11.9 |
| AUCₗₐₛₜ | 1585 | 2021 | 1787 | 2263 | 1914 | 293 | 15.3 |
| (h*ng/mL) | | | | | | | |
| AUC_{Inf} | 1696 | 2180 | 2018 | 2424 | 2080 | 306 | 14.7 |
| (h*ng/mL) | | | | | | | |
| AUC_{_{—}}_{% Extrap}_{_{—}}obs | 6.54 | 7.32 | 11.5 | 6.64 | 8.0 | 2.3 | 29.3 |
| MRT_{Inf}_{_{—}}obs (h) | 1.01 | 1.10 | 1.46 | 1.17 | 1.19 | 0.19 | 16.4 |
| AUCₗₐₛₜ/D | 158 | 202 | 179 | 226 | 191 | 29 | 15.3 |
| (h*mg/mL) | | | | | | | |
| Vₛₛ_{_{—}}obs (L/kg) | 5.96 | 5.05 | 7.24 | 4.83 | 5.77 | 1.09 | 19.0 |

TABLE 7
CCL-104 pharmacokinetic parameters when administered PO

| PK parameters | Mouse 5 | 6 | 7 | 8 | Mean | SD | CV(%) |
|---|---|---|---|---|---|---|---|
| T_{1/2} (h) | 1.72 | 3.26 | 3.38 | 2.08 | 2.61 | 0.84 | 32.0 |
| Tₘₐₓ (h) | 0.500 | 0.250 | 0.250 | 0.250 | 0.313 | 0.125 | 40.0 |
| Cₘₐₓ (ng/mL) | 433 | 462 | 438 | 464 | 449 | 16 | 3.57 |
| AUCₗₐₛₜ (h*ng/mL) | 591 | 467 | 522 | 569 | 537 | 55 | 10.3 |
| AUC_{Inf} (h*ng/mL) | 656 | 659 | 631 | 623 | 642 | 18 | 2.77 |
| AUC_{_{—}}_{% Extrap}_{_{—}}obs | 9.81 | 29.2 | 17.3 | 8.73 | 16.2 | 9.4 | 57.9 |
| MRT_{Inf}_{_{—}}obs (h) | 2.08 | 4.24 | 2.98 | 2.16 | 2.87 | 1.00 | 35.0 |
| AUCₗₐₛₜ/D | 59.1 | 46.7 | 52.2 | 56.9 | 53.7 | 5.5 | 10.3 |
| (h*mg/mL) | | | | | | | |
| F (%) | 31.5 | 24.4 | 30.3 | 30.0 | 29.1 | 3.2 | 10.9 |

TABLE 8
CCL-106 pharmacokinetic parameters when administered IV

| PK parameters | Mouse 1 | 2 | 3 | 4 | Mean | SD | CV(%) |
|---|---|---|---|---|---|---|---|
| Cl_obs (mL/min/kg) | 84.1 | 66.2 | 82.1 | 66.3 | 74.7 | 9.7 | 13.1 |
| T_{1/2} (h) | 1.63 | 2.14 | 2.50 | 2.99 | 2.32 | 0.57 | 24.8 |
| C₀ (ng/mL) | 3719 | 4701 | 5792 | 5062 | 4819 | 862 | 17.9 |
| AUCₗₐₛₜ (h*ng/mL) | 1731 | 2047 | 1630 | 1952 | 1840 | 193 | 10.5 |
| AUC_{Inf} (h*ng/mL) | 1983 | 2519 | 2031 | 2513 | 2261 | 295 | 13.0 |
| AUC_{_{—}}_{% Extrap}_{_{—}}obs | 12.7 | 18.7 | 19.8 | 22.3 | 18.4 | 4.1 | 22.2 |
| MRT_{Inf}_{_{—}}obs (h) | 1.62 | 2.11 | 2.16 | 2.49 | 2.10 | 0.36 | 17.0 |
| AUCₗₐₛₜ/D (h*mg/mL) | 173 | 205 | 163 | 195 | 184 | 19 | 10.5 |
| Vₛₛ_{_{—}}obs (L/kg) | 8.18 | 8.38 | 10.7 | 9.89 | 9.3 | 1.2 | 12.9 |

TABLE 9
CCL-106 pharmacokinetic parameters when administered PO

| PK parameters | Mouse 5 | 6 | 7 | 8 | Mean | SD | CV(%) |
|---|---|---|---|---|---|---|---|
| T_{1/2} (h) | 3.20 | 3.31 | 2.04 | 8.02 | 4.14 | 2.65 | 64.0 |
| Tₘₐₓ (h) | 0.250 | 0.250 | 0.250 | 0.250 | 0.250 | 0.00 | 0.00 |
| Cₘₐₓ (ng/mL) | 233 | 177 | 352 | 127 | 222 | 97 | 43.5 |
| AUCₗₐₛₜ (h*ng/mL) | 314 | 308 | 414 | 226 | 316 | 77 | 24.4 |
| AUC_{Inf} (h*ng/mL) | 383 | 404 | 453 | 479 | 429 | 44 | 10.2 |
| AUC_{_{—}}_{% Extrap}_{_{—}}obs | 18.1 | 23.7 | 8.47 | 52.7 | 25.7 | 19.1 | 74.0 |
| MRT_{Inf}_{_{—}}obs (h) | 3.27 | 4.01 | 2.21 | 10.3 | 4.9 | 3.6 | 73.5 |
| AUCₗₐₛₜ/D (h*mg/mL) | 31.4 | 30.8 | 41.4 | 22.6 | 31.6 | 7.7 | 24.4 |
| F (%) | 16.9 | 16.7 | 20.0 | 12.3 | 16.5 | 3.2 | 19.3 |

To determine potential maximum tolerated dosages, 3.12, 6.25, 10, and 12.5 mg/kg of CCL-106, for 2, 2, 1, and 1 doses respectively, were administered to mice. No toxicity was observed for the 3.12, 6.25, and 10 mg/kg dose. The 12.5 mg/kg administered mouse showed partial toxicity (tremors) for CCL-104 and acute toxicity for CCL-106 (strong tremors and weight loss). Mice were administered high doses of CCL-104 and CCL-106. For CCL-104, a 31.25 mg/kg dose of caused a mouse to stop moving after about 5-10 minutes and have seizure like symptoms; the mouse took another 30-45 minutes to start moving/breathing normally. The mouse was administered daily 31.25 mg/kg doses for two additional days with the same presentation and displayed tail vein toxicity on day 3. At day 4 the mouse did not receive any dose. At each of days 5, 6, and 7, the mouse received a 15.63 mg/kg dose and also had seizure like symptoms but recovered more quickly (around 30 minutes) for each day. A 39.06 mg/kg dose killed a mouse in about 5 minutes. A 46.87 mg/kg dose killed a mouse almost immediately after administration. For CCL-106, a 31.25 mg/kg dose of caused a mouse to stop moving after about 2 minutes and have seizure like symptoms; the mouse took another 30-45 minutes to start moving/breathing normally. The mouse was administered daily 31.25 mg/kg doses for two additional days with the same presentation and displayed tail vein toxicity on day 2. Day 4 the mouse did not receive any dose. At each of days 5, 6, and 7, the mouse received a 15.63 mg/kg dose and also had seizure like symptoms but recovered more quickly (around 30 minutes) each day. A 39.06 mg/kg dose killed a mouse immediately. A 46.87 mg/kg dose killed a mouse immediately.

### Example 5

### Xenograft Studies

Animal housing and environment: Female SCID mice will be housed in individually ventilated cages in a controlled environment (temperature 22±2° C., humidity 30-70%, and 12 h light/12 h dark cycle). Acclimatization of animals: Mice will be acclimated for 2-4 days. For the study, the required number of SCID mice will be allocated and housed separately in the experimental area. Food and water for animals: The mice will have access to commercially available sterile feed pellets and sterile potable water ad libitum. Identification of animals: 4 mice will be housed per individually ventilated cage. Each animal in the cage will be identified by ear punch. These cages will be numbered thus making the animal identification easy. Ethical approvals: All animal experiments will be carried out in accordance with the guidelines of the guide for animal welfare and with the approval of the IACUC WSU. Population per group: 9 mice per group (6 for efficacy and 3 for residual tumor analysis).

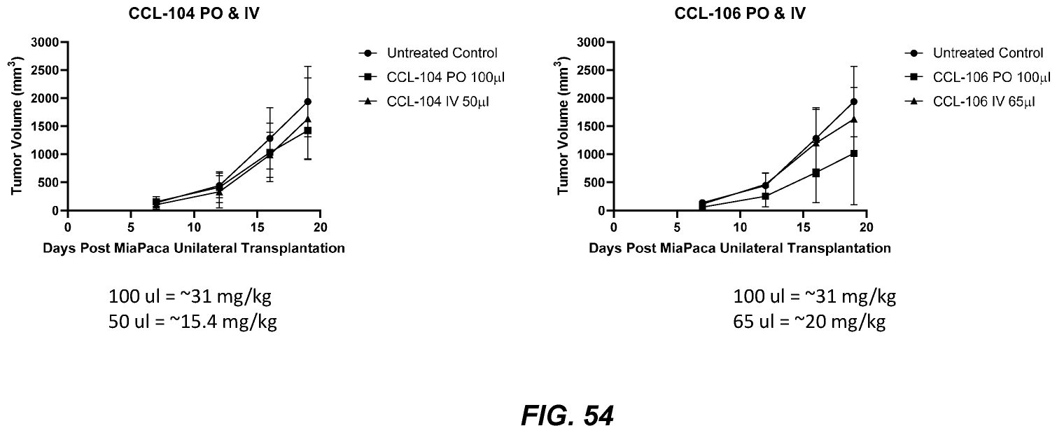

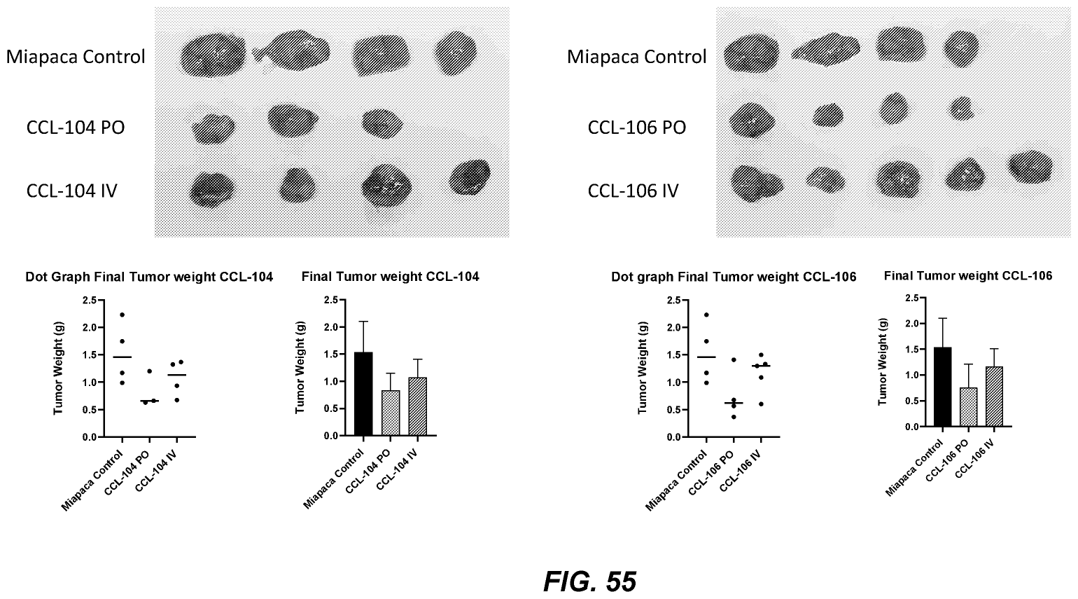

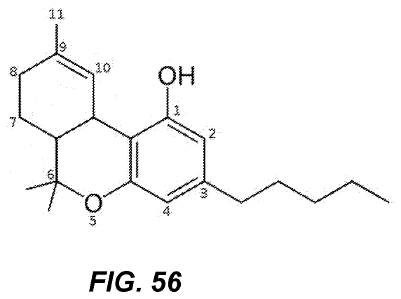

Miapaca cells were xenograft into the mice. 7 days after transplantation, the mice were untreated (administered a vehicle control), administered CCL-104 intravenously (15.4 mg/kg) or orally (31 mg/kg), or administered CCL-106, intravenously (20 mg/kg) or orally (31 mg/kg). At necropsy, tumors were excised for IHC, immunoblotting and RNA isolation for suppression proliferation related signaling molecules. As shown in FIGS. 54-55, the treated mice had smaller tumors compared to the untreated control.

FIG. 55 shows final tumors after treatment in the xenograft study. The dot graph of the final tumor weight for CCL-104 treated tumors showed at 40.6% reduction in PO treated mice and a 30.0% reduction in IV treated mice. The dot graph of the final tumor weight for CCL-106 treated tumors showed at 50.8% reduction in PO treated mice and a 27.0% reduction in IV treated mice. The dot graph of the final tumor weight for CCL-104 treated tumors showed at 40.6% reduction in PO treated mice and a 30.0% reduction in IV treated mice. The bar graph showing the mean of the final tumor weight for CCL-104 treated tumors showed at 45.89% reduction in PO treated mice and a 29.9% reduction in IV treated mice. The bar graph showing the mean of the final tumor weight for CCL-106 treated tumors showed at 50.79% reduction in PO treated mice and a 24.2% reduction in IV treated mice.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Eur J Pharmacol. 2011, 650 (1), 64-71
Scialdone, M. A. (2019). Hydrogenation of cannabis oil (Patent No. 20190030102Δ1).
PCT/US22/74047 A method for preparing hexahydrocannabinol.
J Nat Prod 2021, 84 (11), 2858-2865
U.S. Patent Application Publication No. 2019/0030102

What is claimed is:

1. A compound of formula I or formula II: 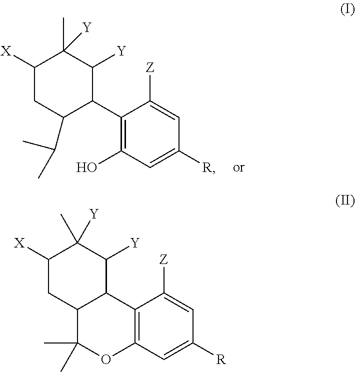
wherein
X is H, halogen, CF₃, or OR²;
R² is CF₃, or C₁₋₆ alkyl;
Y is H, or two adjacent Y are taken together to form a 3-membered heterocylcoalkyl ring, or two adjacent Y are taken together to form a 3-membered cycloalkyl ring optionally substituted with two R³;
Each R³ is independently H or F;
Z is OR⁴;
R⁴ is H, C₁₋₉ substituted or unsubstituted alkyl, C₁₋₉ substituted or unsubstituted alkenyl, C₁₋₉ substituted or unsubstituted alkynyl, OC(O)(CH₂)ₙC(O)O-gemcitabine, or OC(O)(CH═CH)ₙC(O)O-gemcitabine, wherein n is an integer from 1-20;
R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁵; and
R⁵ is H, C₁₋₉ substituted or unsubstituted alkyl, C₁₋₉ substituted or unsubstituted alkenyl, or C₁₋₉ substituted or unsubstituted alkynyl.

2. The compound of claim 1, wherein the compound is further defined as 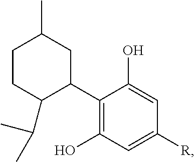
wherein R is a substituted or unsubstituted alkyl group ranging from 1 to 9 carbon atoms.

3. The compound of claim 1, wherein R is CH₃.

4. The compound of claim 1, wherein R is CH₂CH₃.

5. The compound of claim 1, wherein R is CH₂CH₂CH₃.

6. The compound of claim 1, wherein R is CH₂CH₂CH₂CH₃.

7. The compound of claim 1, wherein R is CH₂CH₂CH₂CH₂CH₃.

8. The compound of claim 1, wherein R is CH₂CH₂CH₂CH₂CH₂CH₃.

9. The compound of claim 1, wherein R is CH₂CH₂CH₂CH₂CH₂CH₂CH₃.

10. The compound of claim 1, wherein R is CH₂CH₂CH₂CH₂CH₂CH₂CH₂CH₃.

11. The compound of claim 1, wherein R is CH₂CH₂CH₂CH₂CH₂CH₂CH₂CH₂CH₃.

12. The compound of claim 1, wherein R is C(CH₃)₃.

13. The compound of claim 1, wherein R is CH(CH₃)CH(CH₃)CH₂CH₂CH₂CH₂CH₃

14. The compound of claim 1, wherein R is CH(CH₃)₂.

15. The compound of claim 1, wherein R is CH₂CH(CH₃)₂.

16. The compound of claim 1, wherein R is CH₂CH₂CH(CH₃)₂.

17. The compound of claim 1, wherein R is C(CH₃)₂CH₂CH₂CH₂CH₂CH₂CH₃

18. The compound of claim 1, wherein the compound is further defined as 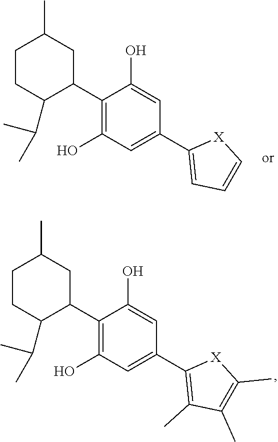
wherein X is N, O, or S.

19. The compound of claim 1, wherein the compound is further defined as 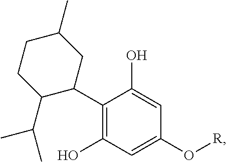
wherein R is C₁₋₄ alkyl-CH═CF₂.

20. The compound of claim 1, wherein the compound is further defined as 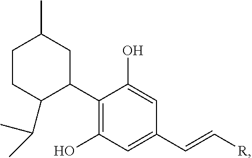
wherein R is a substituted or unsubstituted alkyl group ranging from 1 to 7 carbon atoms.

21. The compound of claim 1, wherein the compound is further defined as 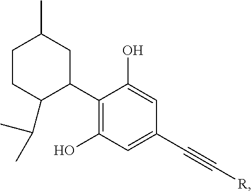
wherein R is a substituted or unsubstituted alkyl group ranging from 1 to 7 carbon atoms.

22. The compound of claim 1, wherein the compound is further defined as

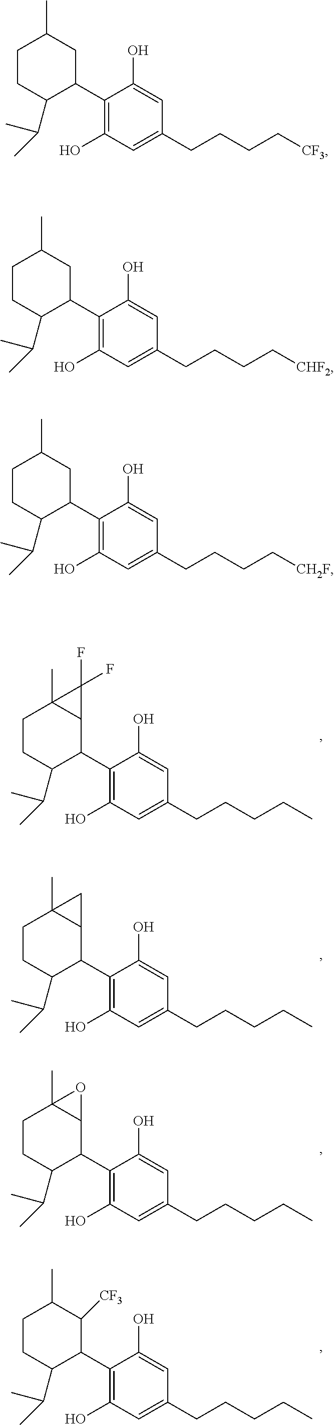

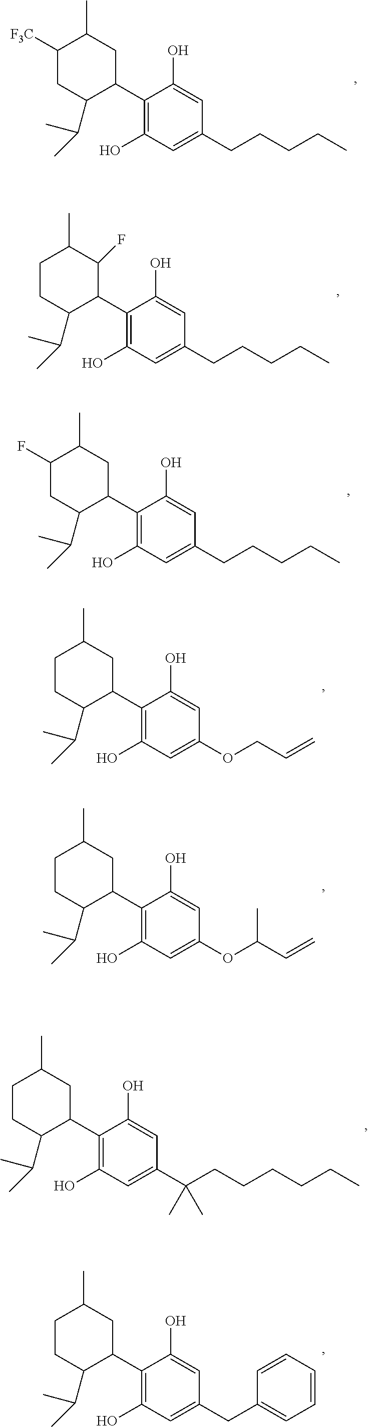

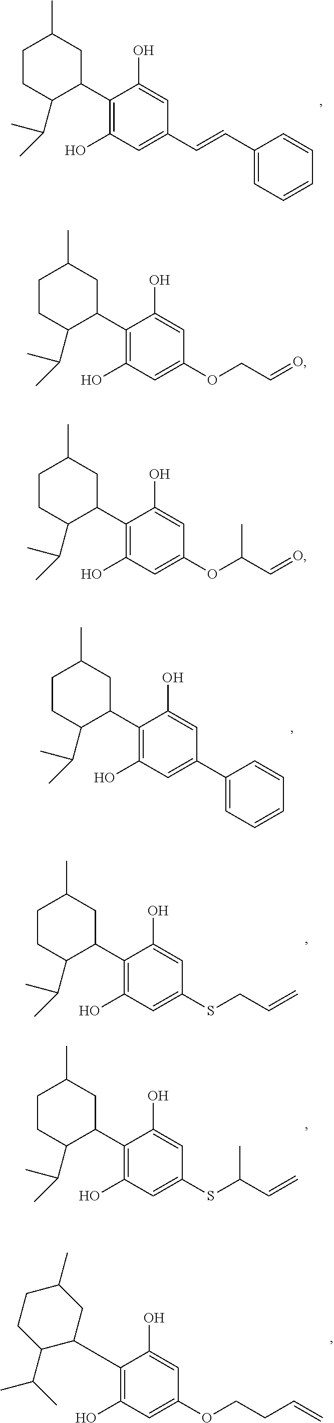

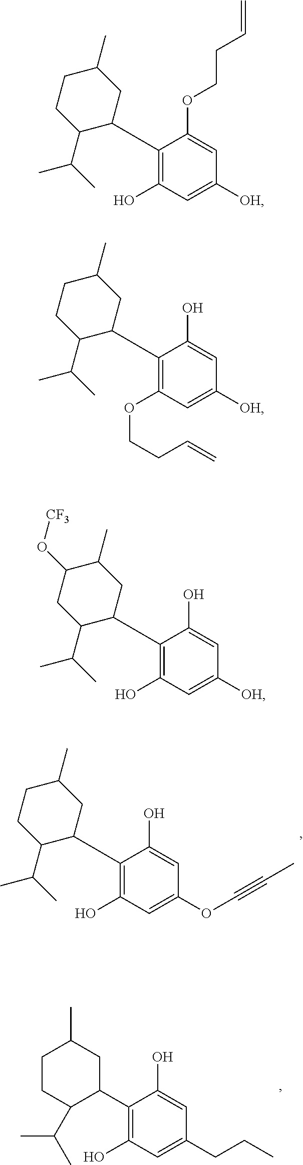

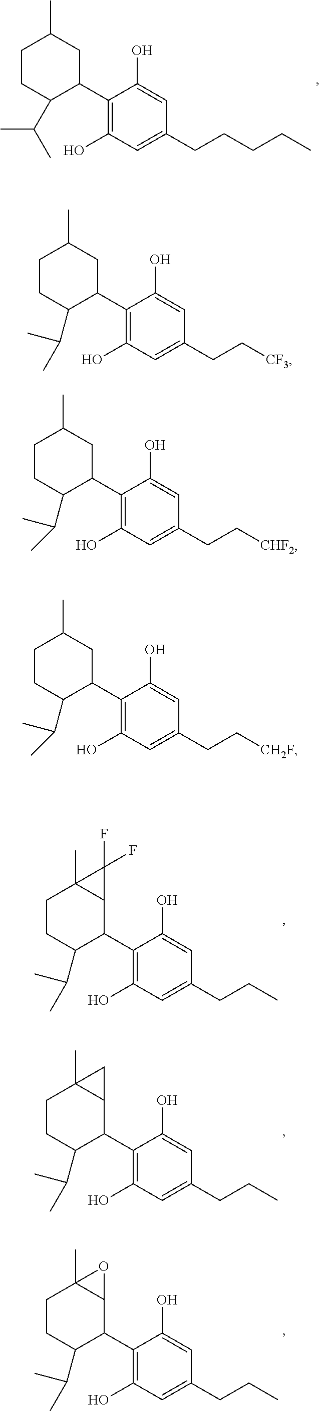

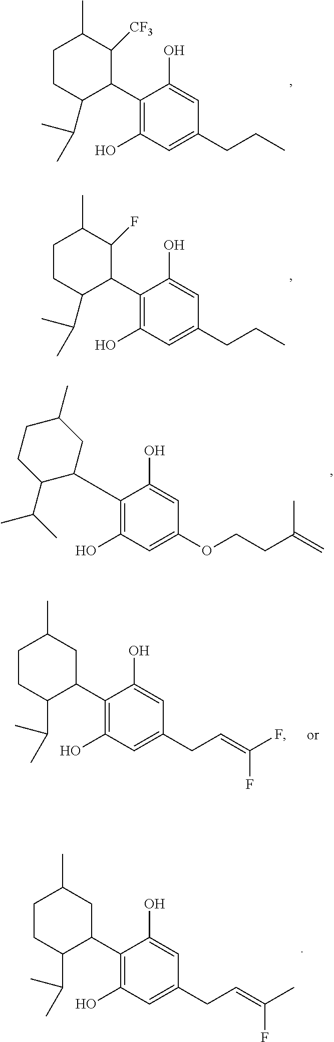

23. A pharmaceutical composition comprising the compound of claim 1 or a derivative or a diastereomer or enantiomer or stereoisomer thereof.

24. The pharmaceutical composition of claim 23, further comprising a pharmaceutically acceptable excipient.

25. The pharmaceutical composition of claim 23, further comprising at least one additional chemotherapeutic.

26. The pharmaceutical composition of claim 25, wherein the at least one additional chemotherapeutic comprises one or more of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride, Olaparib, veliparib, rucaparib, talazoparib, or niraparib.

27. The pharmaceutical composition of claim 25, wherein the at least one additional chemotherapeutic is covalently linked to the compound of Formula I or Formula II.

28. The pharmaceutical composition of claim 27, wherein the at least one additional chemotherapeutic is covalently linked to the compound of Formula I or Formula II via a linker.

29. The pharmaceutical composition of claim 28, wherein the linker is an ester or amino acid linker.

30. The pharmaceutical composition of claim 23, wherein the at least one additional chemotherapeutic is gemcitabine, paclitaxel, or 5-fluorouracil.

31. The pharmaceutical composition of claim 23, wherein the pharmaceutical composition comprises a compound having formula 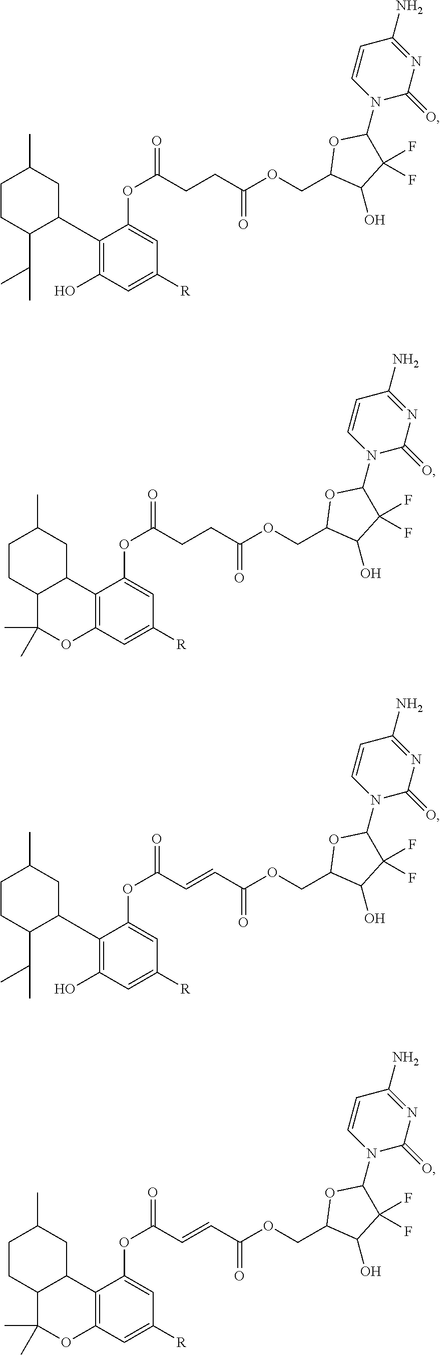 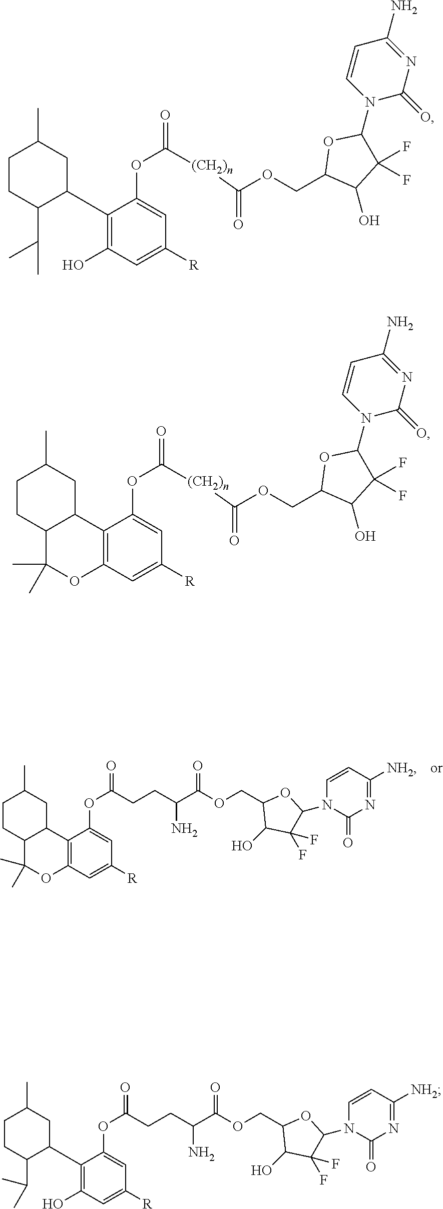
wherein n is an integer between 1 and 10;
wherein R is halogen, C₁₋₉ substituted or unsubstituted alkyl, C₁₋₉ substituted or unsubstituted alkenyl, C₁₋₉ substituted or unsubstituted alkynyl, benzyl, aryl, —S—C₁₋₆ alkyl-CH═CH₂, substituted or unsubstituted pyrrole, substituted or unsubstituted thiophene, substituted or unsubstituted furan, or OR⁶; and
wherein R⁶ is H, C₁₋₉ substituted or unsubstituted alkyl, C₁₋₉ substituted or unsubstituted alkenyl, or C₁₋₉ substituted or unsubstituted alkynyl.

32. A method of treating cancer comprising administering to a subject a therapeutically effective amount of the pharmaceutical composition of claim 23.

33. The method of claim 32, wherein the subject has pancreatic cancer.

34. The method of claim 32, wherein the subject has lung cancer.

35. A method of killing a cancer cell, the method comprising administering to the cancer cell an effective amount of the pharmaceutical composition of claim 23.

36. The method of claim 35, wherein the cancer cell is a pancreatic cancer cell.

37. The method of claim 35, wherein the cancer cell is a lung cancer cell.

38. The method of claim 35, wherein the cancer cell is from a cancer selected from the group consisting of: multiple myeloma, leukemia, alveolar rhabdomyosarcoma, melanoma, lymphoma, astrocytoma, biphasic synovial sarcoma, bladder carcinoma, bone cancer, breast cancer, cecum adenocarcinoma, cervical cancer, cns cancer, colon cancer, colorectal cancer, duodenal adenocarcinoma, embryonal rhabdomyosarcoma, endometrial cancer, epithelioid sarcoma, fibrosarcoma, gastric cancer, signet ring cell gastric adenocarcinoma, gestational choriocarcinoma, glioblastoma, hereditary thyroid gland medullary carcinoma, hypopharyngeal squamous cell carcinoma, invasive ductal carcinoma, liposarcoma, lung cancer, neuroblastoma, osteosarcoma, ovarian cancer, uterine cancer, pancreatic cancer, papillary renal cell carcinoma, prostate cancer, rectal adenocarcinoma, medulloblastoma, renal cancer, testicular embryonal carcinoma, and tongue squamous cell carcinoma.

39. A method of synthesizing the compound of claim 1, the method comprising saturating a double bond between carbons 9 and 10 in a cannabinoid scaffold.

40. The method of claim 39, wherein the cannabinoid scaffold is derived from hemp, Cannabis sativa, Cannabis indica, Echinacea purpurea, Echinacea angustifolia, Acmella oleracea, Helichrysum umbraculigerum, and/or Radula marginata.

41. The method of claim 39, wherein the cannabinoid scaffold comprises cannabinol, tetrahydrocannabinol, cannabidiol, and/or cannabidivarin.

42. The method of claim 39, wherein the cannabinoid scaffold comprises cannabinol, tetrahydrocannabinol, cannabidiol, and/or cannabidivarin, wherein the scaffold is isolated from hemp, Cannabis sativa, Cannabis indica, Echinacea purpurea, Echinacea angustifolia, Acmella oleracea, Helichrysum umbraculigerum, and/or Radula marginata.
